(19) United States
(12) Patent Application Publication
(10) Pub. No.: US 2026/0041888 A1
(43) Pub. Date: Feb. 12, 2026

(54) DYNAMIC RIGIDIZATION METHODS AND APPARATUSES

(71) Applicant: Neptune Medical Inc., Burlingame, CA (US)

(72) Inventors: Alexander Q. TILSON, Burlingame, CA (US); Garrett J. GOMES, San Mateo, CA (US); Mark C. SCHEEFF, San Francisco, CA (US); Jori J. TYTUS, San Mateo, CA (US); Francisco G. LOPEZ, San Mateo, CA (US); Kai POHLHAMMER, San Ramon, CA (US)

(21) Appl. No.: 19/100,929

(22) PCT Filed: Aug. 2, 2023
(86) PCT No.: PCT/US2023/071543

§ 371 Date: Feb. 3, 2025

Related U.S. Application Data
(60) Provisional application No. 63/394,570, filed on Aug. 2, 2022.

(51) Int. Cl. A61M 25/01; A61B 1/005; A61M 25/00

(52) U.S. Cl. CPC A61M 25/0155; A61M 25/0045; A61M 25/0138; A61B 1/0055

(57) ABSTRACT

Described herein are rigidizable apparatuses (e.g., devices, systems, etc.) that may be controlled. e.g., such as by the application of positive and/or negative pressure, to transition between rigid and flexible configurations. These apparatuses may be configured to transition between a highly flexible configuration in which the elongate device may be flexible or floppy and a highly rigid (or selectively rigid) configuration that is many times more rigid than the flexible configuration.

### CLAIM OF PRIORITY

This patent application claims priority to U.S. provisional patent application no. 63/394,570, titled “DYNAMIC RIGIDIZATION METHODS AND APPARATUSES”, filed on Aug. 2, 2022, and herein incorporated by reference in its entirety.

### INCORPORATION BY REFERENCE

All publications and patent applications mentioned in this specification are herein incorporated by reference in their entirety to the same extent as if each individual publication or patent application was specifically and individually indicated to be incorporated by reference.

### BACKGROUND

Surgical devices may include elongate, sometimes tubular structures that include catheters, sheaths, scopes (e.g., endoscopes), wires, overtubes, cannulas, trocars or laparoscopic instruments. The devices can function as a separate add-on device or can be integrated into the body of devices. The devices are inserted into the body so as to access regions within the body, including in some cases forming passages for additional diagnostic and therapeutic medical devices. In some cases it may be beneficial for such elongate medical devices to be rigid or flexible, and in many cases it would be particularly beneficial for these devices to be changed from a flexible configuration into a rigid configuration. There are significant advantages to both highly flexible devices as well significant advantages to rigid devices, however each also have disadvantages. Flexible endoscopes and catheters rely on reaction forces generated by pushing against the tissue of the body cavity being explored to navigate around corners or bends in the anatomy. Flexibility may be problematic when navigating through body regions having highly tortuous passages, areas that are comparatively open, or passages of varying (or large) luminal diameter, where it may be difficult to make reliable contact with the outer diameter of the tube. Further, highly flexible tubes may buckle, prolapse, loop, or may have trouble supporting additional tools or devices. Highly rigid tubes may be difficult to navigate within the body and can cause damage if they are forced through certain anatomical pathways.

Thus, it may be beneficial to provide medical devices that are selectively rigidizable, and which may controllably transition between highly flexible and highly rigid configurations. Although such tools may provide safe, efficient, and precise access to otherwise difficult to reach anatomical locations, it would be beneficial to provide various improvements to rigidizable devices, including improvements that allow the devices to be safer, offer a wider range of flexibility and stiffness, thinner walls, enhance manufacturability, and the ability to function as higher performance combined systems.

Described herein are apparatuses and methods that may address these needs.

### SUMMARY OF THE DISCLOSURE

In general, described herein are rigidizable apparatuses (e.g., devices, accessories, systems, etc.) that may be controlled to transition between rigid and flexible configurations. This transition can occur through multiple means, including by the application of or release of positive and/or negative pressure, links with cables, phase change materials, magnetic materials, electrostatics, nitinol actuation, etc. In some examples, the apparatus may be configured to transition between a highly flexible configuration in which the elongate device (e.g., catheter, tube, rod, etc.) may be flexible or floppy and a highly rigid (or selectively rigid) configuration that is many times (e.g., 2×, 3×, 5×, 7×, 10×, 12×, 15×, 20×, 30×, 40×, 50×, 75×, 100× etc.) more rigid than the flexible configuration. Also described herein are nested sets of two or more devices, of which at least one or more may be rigidizable. These devices may be used to advance or retract the nested set along a tortuous pathway. By selectively rigidizing and un-rigidizing dual rigidizable devices, a shape may be propagated through the tortious pathway.

The rigidizable apparatuses described herein may include rigidizing layers or regions that engage with a compression layer (which may be or may include a bladder) that applies force to the rigidizing layer to rigidize the rigidizing layer or in some cases to de-rigidize (e.g., release from rigidization) the rigidizing layer. In some examples, these rigidizable apparatuses may include a layer that could include a braid, knit, woven, chopped segments, randomly distributed or randomly oriented filaments or strands, engagers, links, scales, plates, segments, particles, granules, crossing filaments, or other materials forming the rigidizing layer.

For example, described herein are rigidizing devices that include a knit material (e.g., knit tube) as all or part of the rigidizing layer. Such a device may include: an elongate flexible tube; a rigidizing layer comprising a knit structure; an inlet configured to attach to a source of pressure; and a compression layer configured to be pushed against the rigidizing layer by a pressure differential from the inlet; wherein the rigidizing device is configured to change between rigid and flexible states by the application of or release of pressure.

In any of these devices the knit layer may be a knit tube. The knit material, which may be referred to herein equivalently as a knit or a knitted material, may be formed of a single fiber or may be knitted from multiple fibers. The fiber forming the knit may be a yarn, a filament, a mono-filament, a plurality of filaments, a strand, a wire, a thread, etc. The fiber may be continuous, in which each of the filament lengths forming the rigidizing layer are part of a single fiber, or they may be broken up into multiple filament lengths. For example, the knit material may be single fiber that is broken/cut at regular or irregular lengths. The knit structure may be configured so that a wale direction of the knit structure extends in a long axis of the flexible tube. Alternatively, the knit structure is configured so that a wale direction of the knit structure is perpendicular to a long axis of the flexible tube.

The knit configuration may be modified in order to optimize the flexibility of these apparatuses in the non-rigidized configuration and/or the rigidity in the rigidized configuration. For example, the knit structure may comprise an average loop length that is two times or greater (e.g., 3×, 4×, 5×, 6×, 7×, 8×, 9×, 10×, 20×, 40×, 60×, 80×, 100× or more) the average loop width. As mentioned, the knit structure forming the rigidizing layer may comprise a knit fiber bundle, a single filament, a bundle of filaments, etc. The material (e.g., filaments) forming the knit structure may be any appropriate material, such as a yarn made of a natural or man-made material, a metal, metal alloy, composite material, polymeric material, natural fiber, etc. In some cases the knit is formed from a fiber, including, for example, aramids (Kevlar, Twaron, Technora), Vectran, UHMWPE (Dyneema or Spectra), Zylon, nylon, polyester, or carbon fiber. In some cases the knit is formed of a composite of multiple materials. In some cases the knit is formed of a metal or multiple metals, including for example, nitinol, a stainless steel alloy, a magnesium alloy, tantalum, cobalt-chromium alloys, etc.

In any of the rigidizing devices described herein the outer layer may be a reinforced outer layer, including but not limited to a coil-reinforced layer. For example, the elongate flexible tube may comprise a coil-reinforced tube. Alternatively, the elongate flexible tube may comprise a non coil-reinforced tube.

Any of these apparatuses may include one or more inlets. For example, an apparatus (e.g., rigidizing device) may include one or more inlets coupled to a proximal end of the flexible elongate tube. The inlet(s) may be coupled at the proximal end region to a source of pressure (e.g. positive pressure or vacuum/negative pressure) to apply a pressure differential to rigidize or to relax, make more flexible, or de-rigidize the apparatus. The inlet(s) may be coupled to input the system at the distal end, including through a feed-line. For example, the inlet may be configured to attach to a source of positive pressure. In some examples the compression layer is configured to be pushed against the rigidizing layer when the positive pressure is applied through the inlet. In some examples the inlet may be configured to attach to a source of negative pressure, and the compression layer may be configured to be pushed against the rigidizing layer when the negative pressure is applied through the inlet.

Any of the apparatuses described herein may also include a second (or more) inlet, such as a secondary inlet, which may be, for example, on the other side of the compression layer than the first (primary) inlet. The secondary inlet could be passive (i.e., a vent), or active (i.e., as a vacuum port, so as to remove mass (for example, air or water) from within a volume so as to provide additional actuation force, or so as to reduce or eliminate the mass from potential inadvertent release within the body). Thereby, for example, a device may simultaneously provide both positive and negative pressure, for example with one force acting on either side of the compression layer, so as to enhance performance, including, but not limited to, rigidization values (e.g., speed of rigidizing/de-rigidizing, pressure applied, etc.).

The compression layer may be any appropriate layer for applying force against the rigidizing layer to rigidize it. In some examples the compression layer is a bladder. The compression layer may be configured to conform against the rigidizing layer. In some examples the compression layer may be configured so as not to conform to the rigidizing layer. For example, the compression layer may be created so that it pushes but does not appreciably deform into the rigidizing layer. The compression layer may be created so that it pushes against and then deforms or distends into the rigidizing layer. In some examples the compression layer comprises an elastomeric (e.g., stretchy) material. In some cases the compression layer is not elastomeric. The compression layer may be plastic. The compression layer may be a plastomer. The compression layer may be a composite structure. For example, the compression layer may be formed of a less-stretchy material that may be an oversized material (e.g., polyethylene terephthalate (PET), nylon, low density polyethylene (LDPE), or a plastomer). Any of these apparatuses may include multiple different rigidizing regions, e.g., along the length of the apparatus, which may be separately or collectively actuated.

The compression layer may be formed by multiple methods. Many bladders are extruded as tubes. Sheet can be created (e.g., extruded, solution cast, blown, etc.) and then heat-sealed or bonded into a tubular structure. Tubes can be created by dipping, e.g., over a mandrel into an elastomer bath or a solvated elastomer bath. A layer may be created by blowing a film. In this case, the film starts out as a bubble of material (typically a plastic or a plastomer, but sometimes also an elastomer) that, with high pressure air behind it, expands or stretches into a tube that is then carried (usually vertically) as it cools while it is diametrically constrained. This approach provides leak-proof quality control and may create a structure that is lower cost and thinner.

The rigidizing device may be configured to have a rigid configuration when positive pressure or negative pressure is applied through the inlet and a flexible configuration when the pressure is not applied through the inlet.

Examples of rigidizable devices including a knit rigidizable member are described in greater detail herein and may provide numerous advantages as compared to other rigidizing members.

Also described herein are apparatuses (e.g., rigidizable devices) having an integrated compression layer and rigidizing layer. In some examples the rigidizing layer may include filament lengths that are within (including but not limited to encapsulated within) a compression material. Deforming the compression material, e.g., by applying positive and/or negative pressure, may transition the rigidizing layer between flexible and rigid configurations.

For example, a rigidizing device may include: an elongate flexible tube; a rigidizing layer comprising an array of filament lengths within a compression material, wherein the array of filament lengths are configured to slide over each other when the compression material is in a first configuration and wherein the array of filament lengths are engaged against each other when the compression material is in a second configuration; an inlet configured to attach to a source of pressure, wherein the compression material is transition between the first configuration and the second configuration by the application by a pressure differential from the inlet to change the rigidizing layer between a rigid state and a flexible state. The first configuration may be an uncompressed configuration and the second configuration may be a compressed configuration. The source of pressure may be a source of positive pressure or a source of negative pressure (e.g., vacuum).

The array of filament lengths may comprise an array of filaments. For example the array of filament lengths can be part of a single filament or may be multiple lengths. In some examples the filament lengths may be part of a single filament, a bundle of filaments, etc. As mentioned above, the filaments may be wire(s), yarn, etc., and the filament material may be any appropriate material, including metal, metal alloys, polymeric material, natural fibers, etc. The filaments may be any appropriate length. In some examples, the filament length may vary and/or may be different lengths, and the filament crossing pattern may be consistent and ordered or it may be more random. For example, the material may be chopped filaments or stainless steel ‘wool’.

In any of these examples the array of filament lengths may be slideably encapsulated within the compression material in the first configuration. Thus, rather than a layer over or under the rigidizing layer, the compression material may surround and/or encapsulate the strands of the rigidizing material. For example, the compression material may comprise an elastomeric material; the filament lengths may be fully encapsulated, or they may be within a construct in which they are held within channels or cavities of the elastomeric material and as the material is deformed, e.g., by applying a positive or negative pressure, the ridigizing layer may be made rigid. In general, the compression material may be any appropriate compressible material. In some examples the compression material may be a lubricious material and/or a lubricious material may be within the channels or chambers holding the rigidizing layer. In some examples the compression material forms a bladder.

In any of these apparatuses the inlet may be configured to couple the source of pressure to a gap between the elongate flexible tube and the rigidizing layer. The elongate flexible tube may be an inner tube and/or an outer tube of the device. Alternatively or additionally, the inlet may be configured to couple the source of pressure to an encapsulation region between the encapsulated filament lengths and the compression material.

The elongate flexible tube may comprise an inner tube. In any of these examples the device may comprise a reinforced outer layer, such as a coil-reinforced outer layer. The elongate flexible tube may comprise a coil-reinforced tube. The rigidizing device may be configured to have a rigid configuration when positive pressure or negative pressure is applied through the inlet and a flexible configuration when the pressure is not applied through the inlet. Alternatively, the rigidizing layer may be configured to be unrigidized in the first configuration when there is no pressure differential between the inlet and atmosphere.

For example, described herein are rigidizing devices comprising: an elongate flexible tube; a rigidizing layer comprising an array of filament lengths within a bladder, wherein the array of filament lengths are configured to move relative to each other when the bladder is in a flexible configuration and, wherein the array of filament lengths are less mobile relative to each other when the rigidizing layer is pressurized against the elongate flexible tube into a more rigidized configuration.

Also described herein are apparatuses having a rigidizing layer formed of multiple lengths of fibers that cross over and under each other and that are configured to rigidize when positive pressure is applied. Because the lengths of filaments cross over and under each other, the application of positive pressure may be particularly effective and may allow a graded response to positive pressure in which the greater the positive pressure, the more rigid that the device may become.

For example, a rigidizing device may include: an elongate flexible tube; a rigidizing layer comprising an array of filament lengths crossing over and under each other and configured to move relative to each other; an inlet configured to attach to a source of positive pressure; a compression layer configured to be pushed against the rigidizing layer by a pressure differential from the inlet to rigidize the rigidizing layer, wherein the rigidizing device is configured to change between rigid and flexible states by the application of or release of pressure. Moving relative to each other may include multiple types, directions, and modes of motion, including sliding, pivoting, shearing, displacing, etc.

As used herein a plurality of filament lengths may be part of a single strand or may be individual strands of filament. The lengths of filaments may be the same size or may be different sizes. For example, the array of filament lengths may comprise a plurality of discrete filaments. The rigidizing layer may be a tube or other shape that is formed of single fiber or multiple fibers (including a single fiber that is broken/cut at regular or irregular lengths). At least some of the filament lengths of the array of filament lengths may be part of a same filament.

In general, the compression layers descried herein include structural layers that may be (but are not limited to) a bladder layer(s) and/or sheets of materials that apply a compressive force on or against the rigidizing layer to rigidize the rigidizing layer, or in some cases to release the rigidizing layer from rigidization. For example, the array of filament lengths may comprise a woven, braided or knit tube. Filaments may be chopped segments, and/or may be sewn.

The array of filament lengths may comprise one or more wires. As mentioned above, the filament lengths may be formed of any appropriate material and may be a single filament, bundles of filaments, e.g., yarn, metal, metal alloys, composite materials, polymeric material, natural fiber, etc.

Any of these apparatuses may include a reinforced inner and/or outer layer, including a coil-reinforced layer. In any of these examples the outer layer is not a coil-reinforced layer, as other outer layers may be used. In some examples the elongate flexible tube comprises a coil-reinforced tube. The elongate flexible tube may comprise a tube that is not coil-reinforced. The elongate flexible tube may comprise a reinforced tube that is not a coil. The elongate flexible tube may comprise a laser cut tube. The elongate flexible tube may comprise a series of linkages. The inlet may be coupled to a proximal end of the flexible elongate tube. The inlet may be configured to attach to a source of positive pressure, further wherein the compression layer is configured to be pushed against the rigidizing layer when the positive pressure is applied through the inlet. The inlet may be configured to attach to a source of negative pressure, further wherein the compression layer is configured to be pushed against the rigidizing layer when the negative pressure is applied through the inlet. In some examples the compression layer comprises an elastomeric layer. In some examples the compression layer comprises a bladder, or multiple bladders, e.g., for multiple rigidizing regions. In any of the apparatuses described herein the bladders may be elastomeric or may not be elastomeric. For example, they may be plastic, a plastomer, or composite. The rigidizing devices described herein may be configured to have a rigid configuration when positive pressure or negative pressure is applied through the inlet and a flexible configuration when the pressure is not applied through the inlet.

Also described herein are apparatuses that are actuated by pressure, including high pressure. In some examples the greater the applied pressure, the more rigid the apparatus will become. For example, described herein are positive pressure (e.g., high pressure) apparatuses in which the compressible member (e.g., bladder) deforms, distending and interdigitating into the wires forming the rigidizing layer. A rigidizing device may include: a flexible inner tube created to or reinforced to withstand a radially compressive load; a flexible outer tube reinforced to withstand a radially tensile load; a rigidizing layer between the inner and outer tubes comprising a plurality of filament lengths crossing over and under each other and configured to move relative to each other; a compression layer configured to deform onto or into the rigidizing layer when a positive pressure is applied to the compression layer, wherein the application of pressure restricts (or in some examples reduces) movement of the plurality of filament lengths, thereby increasing rigidization. For example, as a positive pressure device is pressurized, the positive pressure of the inner tube may drive the reduction of the diameter of the inner tube, and/or may cause it to structurally collapse, including radially collapse or through other forms of collapse.

The device is configured to resist these failures within normal operating pressures. As the positive pressure device is pressurized, the outer tube may experience the positive pressure applied as an expansive or tensile load to the reinforcing wires, nominally expanding its diameter, and, if the reinforcements are undersized, fracturing the reinforcing elements. The device is configured to resist these failures within normal operating pressures.

As mentioned, the deformable compression layer may comprise a bladder. The rigidizing layer may be between the flexible outer tube and the compression layer and wherein the compression layer is configured to press the rigidizing layer into the outer tube when the positive pressure is applied to the compression layer. The rigidizing layer may be between the flexible inner tube and the compression layer and wherein the compression layer is configured to press the rigidizing layer into the inner tube when the positive pressure is applied to the compression layer. In some examples the deformable compression layer comprises an elastomeric layer.

The flexible outer tube may comprise a reinforced (e.g., spiral reinforced, braid reinforced, coil reinforced, etc.) inlaid member(s) and/or layer. Alternatively or additionally, the flexible inner tube may comprise a reinforced layer. The array of filament lengths may comprise a plurality of filaments. These filament lengths may be formed of a single fiber or multiple fibers (including being formed of a single fiber that is broken/cut at regular or irregular lengths). In some examples the array of filament lengths comprises a weave or a braid or a knit. The filament lengths may be ordered or not ordered. The array of filament lengths may comprise one or more wires. For example, the array of filament lengths can be made of a single filament, a bundle of filaments, e.g., yarn, metal, metal alloys, polymeric material, natural fiber, etc.

Any of these apparatuses (e.g., devices) may include one or more inlets that are in fluid communication with the compression layer and are configured to couple to a source of positive pressure. Different inlets may control the application of a pressure differential (e.g., positive and/or negative pressure) to different regions of the apparatus, to allow selective rigidization of different region of the apparatus. The inlet may be coupled to either end of the flexible elongate tube (proximal or distal), or to an intermediate location.

The rigidizing devices described herein may be configured to have a rigid configuration when positive pressure is applied through the inlet and a flexible configuration when the pressure is not applied through the inlet. Alternatively, in some examples the rigidization devices described herein may be configured to have an un-rigidized (flexible) configuration when positive pressure is applied through the inlet and a rigid configuration when the pressure is not applied through the inlet.

The apparatuses described herein may include one or more rigidizing layers that are actuated by the application of positive pressure, including high pressure. In particular, described herein are rigidizing devices having a rigidizing layer formed of a plurality of particles or granules that may move in a loose configuration in the flexible configuration (e.g., when there is no pressure differential relative to atmosphere) but may be rigid when positive pressure is applied, e.g., via a compression layer. When positive pressure is applied, the rigidizing layer may become consolidated or jammed, thereby making the device less flexible or more rigid. For example, a rigidizing device may include: a flexible inner tube; a flexible outer tube; a rigidizing layer comprising a plurality of granules between the inner and outer tubes; an inlet configured to attach to a source of positive pressure; and a compression layer between the inner and outer tubes that is configured to be pushed against the rigidizing layer when a positive pressure is applied through the inlet to compress and rigidize the rigidizing layer.

The granules may be any appropriate size or distribution of sizes (e.g., 1 mm diameter or less, 0.8 mm diameter or less, 0.7 mm diameter or less, 0.6 mm diameter or less, 0.5 mm diameter or less, 0.4 mm diameter or less, 0.3 mm diameter or less, 0.2 mm diameter or less, 0.1 mm diameter or less, 0.05 mm diameter or less, 0.01 mm diameter or less etc.). The granules may be any appropriate material, typically a rigid or semi-rigid material (e.g., polymer, metal, mineral, composite material, etc.). The granules may be formed of biocompatible material. In some cases the granules may be bioresorbable. The granules may be crystalline. The granules may have any appropriate shape. For example, the granules may be round, square, faceted, long, oblong, rectangular, obtuse, etc. In some examples the shape of the granules may be regular. In some examples the shape of the granules may be irregular. The granules may include both regular and irregular shapes and may include a variety of different shapes and/or sizes in the same rigidizing layer. The granules may be enclosed within an enclosure, such as a packet, which may be formed into a cylinder. The packet may be sealed or porous (e.g., may include pores that are smaller than the granules). In some examples the compression layer may contain or may partially contain the granules. The compression layer may actuate, urge, push, or consolidate the rigidizing layer. For example, the compression layer may be the enclosure or part of the enclosure. The compression layer may be a bladder. The compression layer may be an elastomeric layer.

As in any of the examples described herein, the outer tube and/or the inner tube may be or may not be reinforced. It could be a laser cut tube. The laser cut tube could be integrated with a distal bending section, which has a different cut pattern but is part of the same tube. For reinforced versions, for example, the inner and/or outer tube may include a coil reinforcement, for example a material that exhibits high tensile strength. This could be a wire, a polymer, a composite fiber, a yarn made of a natural or man-made material, a metal, a metal alloy, a composite material, a polymeric material, a natural fiber, etc. In some cases it could be a fiber, including, for example, aramids (Kevlar, Twaron, Technora), Vectran, UHMWPE (Dyneema or Spectra), Zylon, nylon, polyester, polyethylene, dacron, polypropylene, fiberglass, basalt, or carbon fiber. In some cases it could be formed of a composite of multiple materials. In some instances it could be formed of a metal, including, for example nitinol, a stainless steel alloy, a magnesium alloy, tantalum, cobalt-chromium alloys, etc.

Any of the apparatuses including granules as part of the rigidizing layer may be configured to change between rigid and flexible states by the application of or release of pressure. Thus, the application of positive pressure may compress the granules, in some examples by driving the compression layer against the granules and the inner and/or outer tube to rigidize the granules, without requiring a vacuum to be applied.

Also described herein are apparatuses (e.g., devices) and methods in which the rigidizing layer comprises a plurality of members (e.g., layers, pieces, parts, sub-layers such as arms, scales, plates, etc.). The application of pressure (e.g., positive pressure) may drive the plurality of sub-layers (e.g., arms, plates, scales, etc.) against the inner (or in some configurations, the outer) tube and/or adjacent sub-layers; as the pressure increases the device may become increasingly rigid. This application of positive pressure may deliver a consolidating force that is substantially higher than that which can be delivered by the one atmosphere of vacuum. Alternatively, in some configurations the device may be configured so that the sub-layers are biased against each other in the un-actuated state (when pressure is not being applied) and the application of positive pressure separates the sub-layers from the inner or outer tube and/or each other, transitioning the device from a rigid state to a flexible state.

For example, described herein are rigidizing devices comprising: a flexible outer tube; a flexible inner tube; a rigidizing layer comprising a plurality of overlapping members between the inner and outer tubes; an inlet configured to attach to a source of positive pressure; and a compression layer between the outer and inner tubes and configured to be pushed against the rigidizing layer when a positive pressure is applied through the inlet to compress and rigidize the rigidizing layer.

The overlapping members may comprise a plurality of overlapping sub-layers (e.g., scales or plates). In some examples the overlapping members comprise a plurality of arms extending from one or more radial attachment sections; the radial attachment sections may extend along the length of the device and the plurality of arms may extend either proximally and/or distally from the radial attachment section. The radial attachment section may be a partial or complete ring. In some examples the overlapping members are radially and longitudinally arranged between the inner and outer tubes. Any number of overlapping sub-layers (e.g., arms, scales, plates, etc.) may overlap with each other. For example, the overlapping members may comprise two or more layers of overlapping members. The overlapping members may interdigitate. For example, in some examples the plurality of overlapping members interdigitate along a length of the rigidizing layer. In the flexible configuration the plurality of overlapping members may be configured to slide over each other, while in the rigid configuration the plurality of overlapping members may be inhibited from sliding over each other. For example, in some cases the plurality of overlapping members may each comprise one or more engagement features between the overlapping members.

Any appropriate compression layer may be used. As mentioned, in some examples the compression layer comprises a bladder. The compression layer may be an elastomeric layer or a non-elastomeric layer. The outer and/or inner tube may comprises be reinforced. For example, the flexible inner and/or outer tube may comprise a coil-reinforce layer. In any of these examples the inlet may be coupled to a proximal end of the flexible elongate tube.

The rigidizing device may be configured to change between rigid and flexible states by the application of or release of pressure (e.g., positive pressure).

In some examples the rigidizing device includes a rigidizing layer with sub-layers (e.g., arms, scales, plates, etc.) that may overlap in either the static or dynamically positioned configuration; in some examples the sub-layers do not overlap in either the static or dynamically positioned configuration. The sub-layers may instead be adjacent to (e.g., overlapping or non-overlapping) each other radially and along the length of the apparatus. For example, a rigidizing device may comprise: a flexible outer tube; a flexible inner tube; a rigidizing layer comprising a plurality of adjacent members arranged radially and longitudinally adjacent to each other between the inner and outer tubes; an inlet configured to attach to a source of positive pressure; and a compression layer between the outer and inner tubes and configured to be pushed against the rigidizing layer when a positive pressure is applied through the inlet to compress and rigidize the rigidizing layer. In some examples these layers may be positioned by spiral wrapping. These layers may be positioned by attaching the scales to a central ‘backbone’ (for example, a wire, or base material connecting the scales). This backbone may be attached at one end and slide at another as the structure is bent or articulated, thereby changing the inner or outer path length.

These layers may be positioned by attaching them centrally to an underlying or over-structure, such that they are in a relatively fixed position, but can still have portions of their geometry that slide over other adjacent elements, such that those relative positions would then be fixed as pressure is applied and layers are consolidated together.

For example, the adjacent members may include a plurality of arms extending from one or more radial attachment sections. The adjacent members may be configured so that they do not overlap (e.g., the adjacent members are non-overlapping), or may overlap only when the device is bent beyond a predetermined angle.

The overlapping members may include two or more rings of adjacent members arranged radially around the device and/or may include two or more rows of adjacent members.

As described above, the compression layer may comprise an elastomeric or non-elastomeric material. In some examples the compression layer comprises a bladder. The outer and/or inner tube may be reinforced. Any of these devices may include one or more inlets. Any of these devices may include a rigidizing device that is configured to change between rigid and flexible states by the application of or release of pressure.

Also described herein are rigidizing devices including a plurality of mating geometric shapes that may engage with each other to rigidize the device when positive pressure is applied (e.g., to the compression layer). For example, described herein are rigidizing devices comprising: a flexible outer tube; a flexible inner tube; a rigidizing layer comprising a plurality of radially engaging members between the inner and outer tubes; an inlet configured to attach to a source of positive pressure; and a compression layer between the outer and inner tubes and configured to be pushed against the rigidizing layer when a positive pressure is applied through the inlet to drive engagement of the radially engaging members.

The plurality of radially engaging members may include interlocking members. For example, the plurality of radially engaging members may comprise a plurality of radially nested members extending along a proximal to distal length. The plurality of radially engaging members may include a plurality of radially compliant members.

In some examples the rigidizing layer may be configured as a woven set. For example, described herein are rigidizing devices comprising: a flexible outer tube; a flexible inner tube; a rigidizing layer comprising a woven layer between the inner and outer tubes; an inlet configured to attach to a source of positive pressure; and a compression layer between the outer and inner tubes and configured to be pushed against the rigidizing layer when a positive pressure is applied through the inlet to compress and rigidize the rigidizing layer. The woven layer may comprise a plurality of filament lengths crossing over and under each other and configured to shear relative to each other. The compression layer may be configured to conform around the plurality of filament lengths to contact the flexible outer layer or the flexible inner layer to prevent shear of the plurality of filament lengths relative to each other when positive pressure is applied through the inlet. The plurality of filament lengths may be separate (e.g., may be discrete lengths of filament that are not part of the same strand or strands of filament). For example, the plurality of filament lengths may be broken or cut into a network of separate strands or sections. This may enhance flexibility, while still allowing rigidization as the individual strands overlap with each other. The individual strands may be of any appropriate length or range of lengths. For example, the individual strands may have a length that is less than the diameter of the tube (e.g., 80% or less, 70% or less, 60% or less, 50% or less, 40% or less, 30% or less, 20% or less, 10% or less, etc., or between about 5% and 90%, between about10% and 80% between about 10% and 70%, etc. of the diameter of the flexible inner tube). The individual strands may have a length longer than the length of the tube (e.g., greater than 1.5×, 2×, 4×, 6×, 8×, 10×, 20×, 40×, 100×, etc.).

Any of the apparatuses described herein may be coupled together to form a nested system that may be configured to coordinate movement (e.g., advancement and retraction) and rigidization to allow navigation through tortuous regions of the anatomy. In general, any of the apparatuses descried herein may be steerable. For example, any of these apparatuses may have steerable distal ends. For example, in any of these apparatuses the distal end region may include one or more linkages. These linkages can be actuated by multiple methods, including by cables, motors, hydraulics, pneumatics, shape memory materials, or EAP (electroactive polymers). They may have one or more wires extending proximally from the distal end region to allow steering of the distal end region. The distal end region may be distal to the rigidizing region, or it may be part of the rigidizing system. It may have the same rigidizing elements as the main rigidizing system, or it may have a different rigidizing elements in this distal region. In general, all or a majority of the length of the elongate body of the device may be rigidizable. In other embodiments, only a portion of the length of the elongate body may be rigidizable.

For example, a nested system may include: a first rigidizing device comprising a plurality of layers, the first rigidizing device configured to be rigidized by applying a pressure differential to drive a compression layer against a rigidizing layer forming at least one layer of the plurality of layers of the first rigidizing device; and a second rigidizing device configured to rigidize, the second rigidizing device nested with the first rigidizing device; wherein the first and second rigidizing devices are configured to translate relative to one another and to alternately rigidize to propagate a shape. The second rigidizing device may be inside the first rigidizing device, or it may be outside the first rigidizing device.

The first rigidizing device and the second rigidizing device may be the same type of ridigizing device (e.g., may each include a knit rigidizing member, etc.) or may be different types of rigidizing devices (e.g., the outer rigidizing device may include a knit rigidizing layer and the inner rigidizing device may include a woven rigidizing layer, etc.). The second rigidizing device may be actuated by an array of methods, including those that do not include the application of or removal of positive or negative pressure.

In general, any of the apparatuses described herein may be configured as tubes, e.g., including a central lumen or multiple lumen (e.g., radially within a flexible inner tube) or may be configured as rods (e.g., without an accessible lumen). In particular, the nested systems described herein may include an outer (e.g., mother) device that is configured as a rigidizing tube, and an inner (e.g., child or daughter) device that is configured as a rigidizing tube or as a rigidizing rod. If it is a rigidizing rod, the inner diameter (i.d.) may be used for payload (for example, the constituents of the inside of a scope, such as electrical cables, steering cables, pressure lines, wash lines, and working channels). In another embodiment, all or a portion of the payload (for example, cables, lines) may be positioned outside of the inner diameter. Alternately, it could be a rigidizing device that does not have an inner coil wound tube, for example, a device in which the compression layer forms the i.d. of the device.

The first rigidizing device and the second rigidizing device may each comprise continuously curved surfaces configured to smoothly slide relative to each other. For example, the outer surface of the inner (e.g., child) device may be configured to be smooth and lubricious, and the inner surface of the outer (e.g., mother) device may be smooth and lubricious. Both of these devices may be configured to avoid or prevent wrinkling of the surface against which it may slide (e.g., the inner surface of the outer device and/or the outer surface of the inner device), even when applying pressure to rigidizing the device or release the device from rigidization, and/or in bending.

For example, the first rigidizing device and the second rigidizing device may each comprise a pressurized coil-wound tube configured to form continuously curved surfaces to smoothly slide relative to each other in both the rigidized and un-rigidized configuration. The compression layer (e.g., of the first rigidizing device and/or both rigidizing

devices) may comprise a bladder. The rigidizing layer may comprise a plurality of filament lengths crossing over and under each other and configured to shear relative to each other. In some examples the rigidizing layer may comprise a braided layer, a knit layer, a woven layer, etc. (any of the rigidizing layers described herein). As mentioned, at least one of the first rigidizing device and the second rigidizing device may comprises a steerable or articulated distal end region comprising a plurality of linkages. These could be different types of linkages, including elements of a laser cut tube, discrete linkages, or a tube that has high propensity for bending. At least the first rigidizing device is configured to be rigidized by the application of pressure, either positive or negative. In some examples the second rigidizing device is configured to be nested within the first rigidizing device (alternatively, the first rigidizing device is configured to be nested within the second rigidizing device). Any of these apparatuses may include a controller and actuators configured to coordinate and manipulate the alternating rigidization of and movement of the first rigidizing device and the second rigidizing device.

Any of the nested systems described herein may include a rigidizable device including a knit as the rigidizing layer. For example, described herein are nested systems including: a first rigidizing device comprising a plurality of layers, the first rigidizing device configured to be rigidized by applying a pressure differential to drive a compression layer against a knit rigidizing layer forming at least one layer of the plurality of layers of the first rigidizing device; and a second rigidizing device configured to rigidize, the second rigidizing device nested with the first rigidizing device; wherein the first and second rigidizing devices are configured to translate relative to one another and to rigidize to propagate a shape along the nested system.

As described above, the knit rigidizing layer may comprise a knit tube. In some examples the knit rigidizing layer is configured so that a wale direction of the knit structure extends in a long axis of the flexible tube. Alternatively, the knit rigidizing layer may be configured so that a wale direction of the knit structure is perpendicular to a long axis of the flexible tube (or at an angle relative to the long axis). The knit rigidizing layer may comprise an average loop length that is greater than two times an average loop width. The knit rigidizing layer may comprise a knit fiber bundle.

In any of these examples the first rigidizing device and the second rigidizing device may each comprise continuously curved surfaces configured to smoothly slide relative to each other. The first rigidizing device and the second rigidizing device may each comprise a pressurized coil-wound tube configured to form continuously curved surfaces to smoothly slide relative to each other. As mentioned above, the compression layer may comprise a bladder. At least one of the first rigidizing device and the second rigidizing device may comprises a steerable distal end region comprising a plurality of linkages; for example, the second rigidizing device may include a steerable distal end region including a plurality of linkages that may be actuated by one or more actuation methods (including wires or tendons, which would extend a length of the device). Alternatively or additionally, the distal end region may be steerable by hydraulics, including one or more motors at the distal end region, etc. motors at the distal end, hydraulics, etc.).

The first rigidizing device may be configured to be rigidized by the application of positive pressure. The second rigidizing device may be configured to be nested within the first rigidizing device.

Any of these nested apparatuses may include a controller configured to coordinate the alternating rigidization of the first rigidizing device and the second rigidizing device.

Any of these nested apparatuses may include an actuator or actuators configured to manipulate the devices, in conjunction with signals from a controller and driven by signals from a user input device.

Any of the apparatuses described herein may include a magnetically and/or electrostatically actuated rigidizing device. For example, described herein are nested systems, comprising: a first magnetically rigidizing device configured to be rigidized by applying a magnetic and/or electric field; and a second rigidizing device configured to rigidize, the second rigidizing device nested with the first rigidizing device; wherein the first and second rigidizing devices are configured to translate relative to one another and to alternately rigidize to propagate a shape along the nested system. The first magnetically rigidizing device may comprise a magnetorheological material. Alternatively, the first magnetically rigidizing device may comprise an electroheological material.

As mentioned above, the first rigidizing device and the second rigidizing device may each comprise continuously curved surfaces configured to smoothly slide relative to each other in both the rigidized and un-rigidized configuration. The second rigidizing device may be configured to be nested within the first rigidizing device, or the first rigidizing device may be configured to be nested within the second rigidizing device. Any of these apparatuses may include a controller configured to coordinate the alternating rigidization of the first rigidizing device and the second rigidizing device.

Any of the apparatuses described herein may be configured so that either or both the inner and outer tubes forming the rigidizable device (e.g., inner elongate tube, outer elongate tube) may be reinforced. These tubes may be reinforced by including one or more coils (e.g., helically wound coils) providing radial stiffness and strength without significantly reducing bending flexibility.

Further, any of the apparatuses described herein may be configured so that the inner and/or outer tubes have a different softness (e.g., durometer) on the inner surface as compared to the outer surface. The outward surfaces of the device may be impacted by other devices, or by anatomy. As such, the puncture or scratch resistance is this surface is very important. The inward surfaces can be the surfaces against which the rigidizing layer is forced against or reacted. In this location, proper softness is important for enhanced flexibility, as well as creating the surface into which the rigidizing layer is forced. The modulation of the hardness effectively serves to modulate how the rigidizing layer embeds or distorts under pressure, thereby being a key driver of rigidization values. Surprisingly, the inventors have found the different needs of the different layers are optimized by modulating the material, and its durometer, in each specific location. For example, results can be more optimized by fabricating the outer layers from of a material that is higher durometer including for higher scratch and puncture resistance and by fabricating the inwards layers of the tubes from a lower durometer material including for rigidization range maximization. In any of these apparatuses the outer tubes may be configured so that the outer part or outer layer of the tube is scratch resistant while the inner part or inner surface is softer (e.g., has a lower durometer). This may be used with or without internal reinforcement.

For example, an elongate rigidizing device may include: an inner elongate tube; an outer elongate tube including an inner region, a reinforcing member (e.g., a radially reinforcing member), and an outer region, wherein the inner region has a durometer lower than the durometer of the outer region; a rigidizing layer; an inlet configured to supply positive pressure between the inner elongate tube and the outer elongate tube; a compression layer configured to push the rigidizing layer against the outer elongate tube when a positive pressure is applied through the inlet, wherein the rigidizing device is configured to change between rigid and flexible states by the application of or release of the positive pressure. The outer region may be an external surface of the device, and the inner region may be an inner surface of the outer tube, against which the rigidizing layer and/or compression layer may contact. The outer region may have a durometer of between about 70 A on the Shore A scale and 80 D on the Shore D scale. The inner region may have a durometer of between about 30 A and about 90 A on the Shore A scale.

The reinforcing member may comprise a wound coil (e.g., wire, ribbon, filament, etc.). The reinforcing member may be helically wound around the tube, and in some examples may be referred to as a radially reinforcing member. The wound coil may be between the inner region and the outer region. The coils may be single or multiple. In some examples the wound coil is laminated between the inner region and the outer region. The compression layer may be configured to push the rigidizing layer against the inner layer of the outer elongate tube when a positive pressure is applied through the inlet.

The rigidizing layer may correspond to any of those described above. For example, the rigidizing layer may comprise a plurality of filament lengths crossing over and under each other and configured shear relative to each other. The compression layer may comprise an elastomeric layer and/or may be or may be configured as a bladder.

In any of these apparatuses the inner tube of the rigidizable apparatus may be configured to have a different durometer on the outer region of the tube as compared to the inner region of the tube. For example, an elongate rigidizing device may include: an outer elongate tube; an inner elongate tube including an inner region, a reinforcing member, and an outer region, wherein the outer region has a durometer higher than the durometer of the inner region; a rigidizing layer; an inlet configured to supply positive pressure between the inner elongate tube and the outer elongate tube; a compression layer configured to push the rigidizing layer against the inner elongate tube when a positive pressure is applied through the inlet, wherein the rigidizing device is configured to change between rigid and flexible states by the application of or release of the positive pressure. For example, the outer region may have a durometer of between about 70 A on the Shore A scale and about 80 shore D on the Shore D scale. The inner region may have a durometer of between about 30 A and about 90 Shore A on the Shore A scale.

In some examples the elongate rigidizing device includes: an outer elongate tube; an inner elongate tube including an inner region, a reinforcing member, and an outer region, wherein the outer region has a durometer higher than the durometer of the inner region; a rigidizing layer; an inlet configured to supply negative pressure between the inner elongate tube and the outer elongate tube; the outer tube configured to push the rigidizing layer against the inner elongate tube when a negative pressure is applied through the inlet, wherein the rigidizing device is configured to change between rigid and flexible states by the application of or release of the negative pressure. For example, the inner region may have a durometer of between about 30 A and about 90 A on the Shore A scale and/or the outer region may have a durometer of between about 70 A on the Shore D scale and about 80 Shore D on the Shore D scale. The reinforcing member may include a wound coil. The wound coil may be between the inner region and the outer region (in some examples, laminated between the inner and outer regions). The compression layer may be configured to push the rigidizing layer against the outer elongate tube when a positive pressure is applied through the inlet.

As mentioned, any of the rigidizing layers may be used in these apparatuses, including a rigidizing layer comprising a plurality of filament lengths crossing over and under each other and configured shear relative to each other. The compression layer may comprise an elastomeric layer or a non-elastomeric layer and in some examples is configured as a bladder.

Any of these apparatuses may also include a torsional stiffening layer. In particular, the high-pressure devices described herein may include a torsional stiffening layer. For example, a rigidizing device may include: a flexible inner tube configured to provide torsional stiffness, wherein the flexible inner tube comprises a first coil wire and also a torsional braid; a flexible outer tube; a rigidizing layer between the inner and outer tubes; an inlet configured to attach to a source of positive pressure; a compression layer configured to be pushed against the rigidizing layer when a positive pressure is applied through the inlet, wherein the rigidizing device is configured to have a rigid configuration when positive pressure is applied through the inlet and a flexible configuration when positive pressure is not applied through the inlet.

The first coil wire may comprise a single or multiple flat wire that is helically wound as part of the flexible inner tube. The wires may be wound in the same direction, or counter-wound. They may be separate wires, or one continuous wire. In any of these examples the torsional braid may comprise a plurality of filaments. For example, it might include 2, 3, 4, 5, 6, 7, 8, 9 or 10 parallel filaments per bundle within the braid. The torsional braid may have a braid angle of greater than about 30 degrees (e.g., 30 degrees or more, 35 degrees or more, 40 degrees or more, 45 degrees or more, 50 degrees or more, 55 degrees or more, etc.).

In any of these apparatuses the first coil wire and the torsional braid may be uncoupled to each other. Alternatively, they may be coupled (including through the use of the matrix material) to each other at intermittent locations or discrete regions along the length of the flexible inner tube. For example the first coil wire and the torsional braid may be coupled to each other between every 30 720 degrees of the helically wound coil wire (e.g., between every 60-720 degrees, between every 90-720 degrees, between every 180-720 degrees, between every 270-720 degrees, between every 1-2 turns, between every 1-5 turns, etc.).

In any of these examples, the first coil wire and the torsional braid may be encapsulated within a material (e.g., an elastomeric material). For example, the material may be an elastomeric or polymeric material.

All of the methods and apparatuses described herein, in any combination, are herein contemplated and can be used to achieve the benefits as described herein.

### BRIEF DESCRIPTION OF THE DRAWINGS

A better understanding of the features and advantages of the methods and apparatuses described herein will be obtained by reference to the following detailed description that sets forth illustrative embodiments, and the accompanying drawings of which:

FIG. 1A is a section through an elongate rigidizable device that may be rigidized by the application of negative pressure.

FIG. 1B is an enlarged view showing one example of the arrangement of layers within the elongate rigidizable device of FIG. 1A.

FIG. 2A is a section through an elongate rigidizable device that may be rigidized by the application of positive pressure.

FIG. 2B is an alternative sectional view showing one example of the arrangement of layers within the elongate rigidizing device of FIG. 2A.

FIG. 3 illustrates an example of a section through one example of a nested pair of rigidizable elongate devices, arranged as a mother (outer) (or, for example, a catheter or overtube) and child (inner) (or, for example, a catheter or endoscope) pair.

FIGS. 4A-4H illustrate an example of a method of operating a nested pair of rigidizing elongate devices that may selectively rigidize and un-rigidize to propagate a shape through a tortious pathway.

FIG. 5A shows an example of a rigidizable device including a knit rigidizing layer, shown with the knit rigidizing layer exposed.

FIG. 5B shows an example of a rigidizable device such as the one shown in FIG. 5A with the outer layer(s) covering the knit rigidizing layer.

FIG. 5C is an enlarged view of one example of a knit.

FIG. 5D shows a section view through an example of a knit over an inner tubular member.

FIG. 6A shows an example of a weft knit.

FIG. 6B shows an example of a warp knit.

FIG. 6C shows an example of a knit material formed of a single continuous filament.

FIG. 7A is an example of a woven rigidizing layer formed of filament; this woven rigidizing layer may be used as part of a rigidizable device as described herein.

FIG. 7B is an example of a woven rigidizing material formed of monofilaments that are woven together; this woven rigidizing layer may be used as part of a rigidizable device as described herein.

FIG. 7C shows another example of a woven material.

FIGS. 8A and 8B show examples of braided material that may be used as (or as part of) a rigidizing layer of a rigidizable device as described herein. FIG. 8B shows a braided layer that is discontinuous.

FIGS. 9A-9B schematically illustrate rigidizing of a pressure-driven rigidizable device.

FIGS. 10A-10C schematically illustrate an example of a rigidizable device having an integrated compression layer and rigidizing layer. FIG. 10B shows the rigidizable device of FIG. 10A rigidized by the application of positive pressure in a first gap region. FIG. 10C shows the rigidizable device of FIG. 10A rigidized by the application of positive pressure in a second gap region.

FIG. 11A schematically illustrates a partial cut-away view of a rigidizable device having a rigidizing layer that is formed of a plurality of adjacent segments with deformable members.

FIGS. 11B and 11C shows an enlarged perspective and side views, respectively, of one example of a segment of a rigidizable device as shown in FIG. 11A.

FIG. 11D shows a longitudinal section through a portion of the rigidizable device of FIG. 11A.

FIG. 11E is another example of a longitudinal section through the rigidizable device of FIG. 11A.

FIG. 11F is an example of a transverse section through the rigidizable device of FIG. 11A.

FIG. 11G is a perspective view of a rigidizable device similar to that shown in FIG. 11A.

FIG. 12A-12D schematically illustrate another example of a rigidizable device having a rigidizing layer that is formed of a plurality of adjacent segments with deformable members. FIG. 12A-12B shows cut-away sectional and partially exploded views of the rigidizable device. FIG. 12C shows an enlarged view of a segment of the device of FIGS. 12A-12B. FIG. 12D is a section view through the device of FIGS. 12A-12B

FIG. 13A-13B schematically illustrates another example of a rigidizable device having a rigidizing layer that is formed of a plurality of segments. FIG. 13A shows with the outer layers removed, showing the helically arranged segments. FIG. 13B is a section through the device.

FIGS. 14A-14D illustrate an example of a rigidizable device having a rigidizing layer formed of a plurality of sub-layers (e.g., scales). FIG. 14A shows an example of a device with the outer layers removed, showing the rigidizing layer and inner layer. FIG. 14B illustrates one example of the sub-layers (scales) that may be used to form the rigidizing layer. FIG. 14C schematically illustrates a cross-section through one example of a rigidizing layer and inner layer. FIG. 14D schematically illustrates an example of section through a rigidizable device (e.g., a tip region of a rigidizable device) including a rigidizable layer formed from a plurality of sub-layers (e.g., scales).

FIG. 15A shows a schematic sectional view through a portion of a rigidizing layer comprising a plurality of overlapping members.

FIG. 15B is a schematic sectional view through a portion of a rigidizable device including the rigidizing layer shown in FIG. 15A.

FIG. 15C schematically illustrates rigidization of the portion of the rigidizable device shown in FIG. 15B.

FIG. 16A schematically illustrates an example of a section through a portion of a rigidizable device including a rigidizing layer comprising a plurality of granules.

FIG. 16B shows the section of FIG. 16A when positive pressure is applied to rigidize the rigidizing layer.

FIG. 16C is an enlarged view of region C of FIG. 16A.

FIG. 16D is an enlarged view of region D of FIG. 16B.

FIG. 17A illustrates a cross-sectional view of a portion of a rigidizable apparatus that provides enhanced torsional stiffness.

FIG. 17B is a section through another example of a rigidizable apparatus that provides enhanced torsional stiffness.

FIG. 18 is a section through an example of a portion of a rigidizing apparatus having a plurality of crossing filament lengths or strands arranged within the body of the apparatus as well as a reinforced inner layer and a reinforced outer layer.

FIG. 19 is a section through an example of a portion of a reinforced inner layer with different durometer regions.

FIGS. 20A-20N schematically illustrate examples of sections through various arrangements of rigidizable devices as described herein.

FIGS. 21A and 21B illustrate an example of a robotic system with high torsional stiffness.

FIG. 22 illustrates an example of a robotic system including an external working channel sleeve apparatus as described herein.

### DETAILED DESCRIPTION

The rigidizable apparatuses and methods described herein may be part of a medical access system for diagnosing and treating regions of the body that are otherwise hard to access and operate within, particular during minimally or non-invasive procedures. In particular, these methods and apparatuses may be used in highly tortuous and/or unsupported regions of the body. These methods and apparatuses may be used in combination with, and/or may modify and improve the rigidizable devices and methods of using them described in U.S. patent Ser. No. 11,135,398 (titled “DYNAMICALLY RIGIDIZING COMPOSITE MEDICAL STRUCTURES”), U.S. patent application Ser. No. 17/604,203 (also titled “DYNAMICALLY RIGIDIZING COMPOSITE MEDICAL STRUCTURES”), PCTUS2021024582 (titled “LAYERED WALLS FOR RIGIDIZING DEVICES”), PCTUS2021034292 (titled “RIGIDIZING DEVICES”), PCTUS2022014497, titled “DEVICES AND METHODS TO PREVENT INADVERTENT MOTION OF DYNAMICALLY RIGIDIZING DEVICES,” PCTUS2022019711, titled “CONTROL OF ROBOTIC DYNAMICALLY RIGIDIZING COMPOSITE MEDICAL STRUCTURES,” U.S. provisional patent application 63/265,934, “METHODS AND APPARATUSES FOR REDUCING CURVATURE OF A COLON,” U.S. provisional patent application 63/296,478, titled “RECONFIGURABLE STRUCTURES,”

U.S. provisional patent application 63/308,044, “DYNAMICALLY RIGIDIZING COMPOSITE MEDICAL STRUCTURES,” U.S. provisional patent application 63/324,011, “METHODS AND APPARATUSES FOR NAVIGATING USING A PAIR OF RIGIDIZING DEVICES, U.S. provisional patent application 63/342,618, “EXTERNAL WORKING CHANNELS FOR ENDOSCOPIC DEVICES,” U.S. provisional patent application 63/335,720, “HYGIENIC DRAPING FOR ROBOTIC ENDOSCOPY,” and U.S. provisional patent application 63/332,686, “MANAGING AND MANIPULATING A LONG LENGTH ROBOTIC ENDOSCOPE,” each of which is herein incorporated by reference in its entirety.

Rigidizing apparatuses as described herein may be configured to rigidize when negative pressure and/or positive pressure is applied. These rigidizing apparatuses as described herein may be used in conjunction with other rigidizing devices that rigidize with other methods, including those that do not rely upon the application of positive or negative pressure. For example, a rigidizing device may be configured to include multiple layers arranged into an elongate catheter-like body. The device may include a handle or other manipulator and may include a connection to one or more pressure sources. Applying pressure from the pressure source may be controlled by multiple methods, including operation of a handle or an electronically controlled device. Control may result in a pressure differential that causes the device to transition between a highly flexible configuration, allowing the tubular body to readily bend, when steered or otherwise guided (e.g., over a guidewire, etc.), and one or more (e.g., a continuum) of rigid configurations. In some examples, particularly (but not exclusively) in reference to apparatuses that rigidize based on the application of positive pressure, the rigidity of the elongate body is proportional to the applied pressure differential, so that the greater the pressure differential, the more rigid the device may become over at least a range of pressure differential values.

In general, these apparatuses may include multiple layers, including a rigidizing layer and at least one of an outer or inner layer. Many of these examples also include a compression layer that may engage with the rigidizing layer, and in some examples the apparatus may include a combined rigidizing layer/compression layer. Described herein are rigidizing layers that may be particularly well suited to rapid and precise actuation over a variety of pressures, including in particular positive pressures (e.g., high positive pressures, i.e., atm of about 2 or more, 4 or more, 6 or more, 8 or more, 10 or more, 15 or more, 20 or more, 30 or more, etc.). Any of these apparatuses may also be configured so that at least some of the inner and/or outer layers making up the rigidizable device have different durometers on the inner and outer portion of either the inner or outer layers. Also described herein are apparatuses and methods including nested sets of rigidizable apparatuses, which may include any of these rigidizable devices. Any of these apparatuses may include one or more torsional enhancing layers for improving torsional control, particularly when included as part of a nested pair of rigidizable devices (e.g., as part of the inner, or child, device).

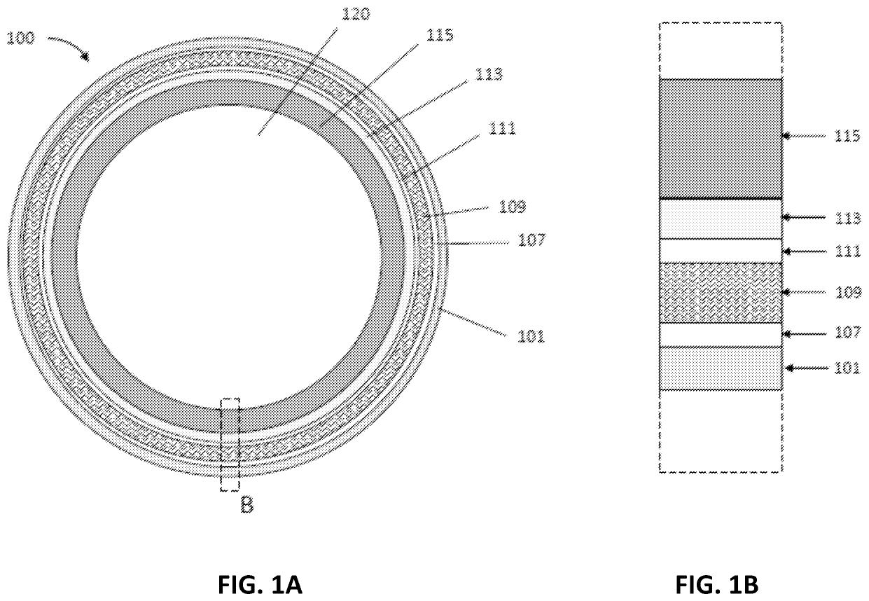

FIG. 1A illustrates an example of a transverse section through an elongate rigidizing device, showing the arrangements of the many layers that may be included. In this example the rigidizable device 100 is configured to be actuated by the application of a negative pressure (e.g., vacuum). The device 100 shown includes an inner layer (115) that may be reinforced (e.g., by including one or more reinforming members, such as a helically arranged strip, ribbon or wire), an optional slip layer (113), a gap (111), a rigidizing layer (109), configured in this example as a braid layer, a second gap (107) and an outer layer (101). In some examples a vacuum may be applied between the outer layer and the inner layer to rigidize. For example, a port configured to couple to the source of negative pressure may be located at the proximal end of the device and may be in fluid communication with the gap region 107 between the flexible outer layer 101 and the rigidizing layer 109, e.g., braided layer. Thus, in this example the outer layer may act as a compression layer. FIG. 1B shows a section through one wall region B of the cylindrical-shaped body of the device. Applying suction may allow the outer layer 101 to be drawn onto the rigidizing layer, causing it to rigidize, limiting or preventing bending of the device.

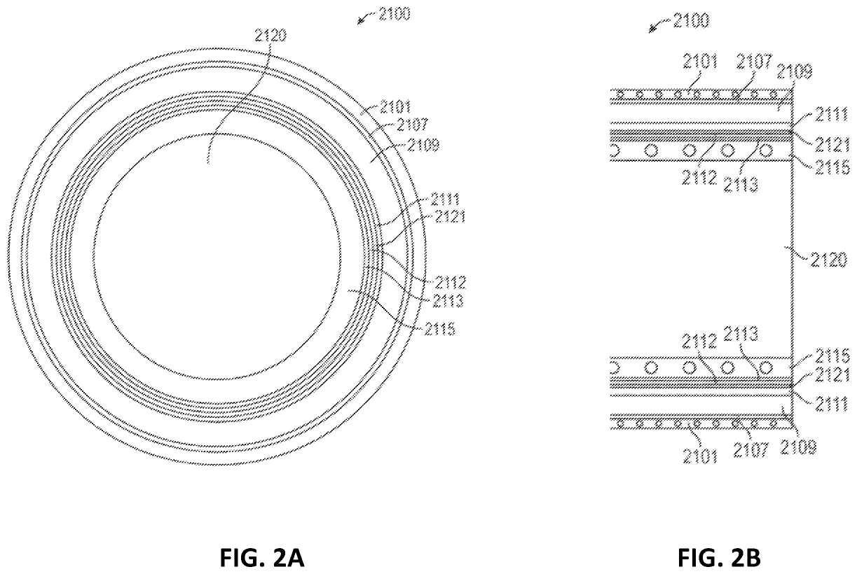

Another example of a rigidizable device is shown in FIGS. 2A-2B. In this example the device may also be an elongate, e.g., catheter or tubular-shaped device similar to that in FIGS. 1A-1B but may be rigidized by the application of positive pressure. For example, FIG. 2A shows a section transverse to the long axis of an elongate rigidizable device. In this example, the layers forming the device are arranged so that an inner reinforced layer 2115 is the most radially-inward layer and may be reinforced, e.g., by a helically wound ribbon, strip, cable, etc. The device may also include an optional slip layer 2113 which may reduce the friction between the inner layer and the more radially-outward layers. The slip layer may be a powder, or it may be a lubricious layer or a layer of lubricious material. A first gap 2112 layer is shown separating the inner layer 2115 and/or the slip layer 2113 from a compression layer, configured in this example as a bladder layer 2121. A second (or intermediate) gap layer 2111 spaces the bladder layer from the rigidizing layer 2109, shown in this example as a braid layer. A third gap layer 2107 is positioned between the rigidizing layer and an outer layer 2101. The outer layer in this example (similar to the inner layer 2115) is reinforced, for example, by a helically wound filament, wire, fiber, band, etc. Although not shown, when actuated by the application of positive pressure between the compression (e.g., bladder) layer and the inner layer, the bladder layer may push the braid layer into the outer layer to rigidize the rigidizing layer.

Both examples of a devices shown in FIGS. 1A-1B and 2A-2B may include additional optional layers or components. Further, the compositions of the rigidizing layers may be modified in order to improve performance. In particular the rigidizing layer maybe modified to include structures (e.g., knits, wovens, braids, scales, plates, arrays of filaments, granules, and combinations thereof, etc.) that may enhance or improve performance. Rigidizing elements may be used as one type alone, or in conjunction with other rigidizing elements. In some examples the inner and/or outer layers may be modified to enhance or improve performance, including the addition of torsional control components, and/or modulating the durometer of the inner and outer regions of these layers.

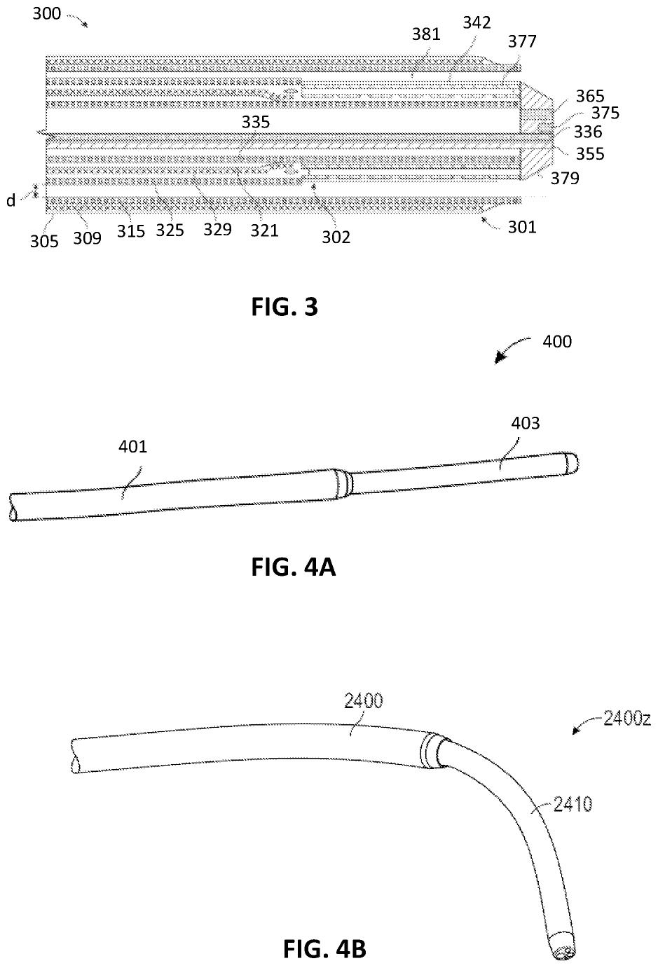

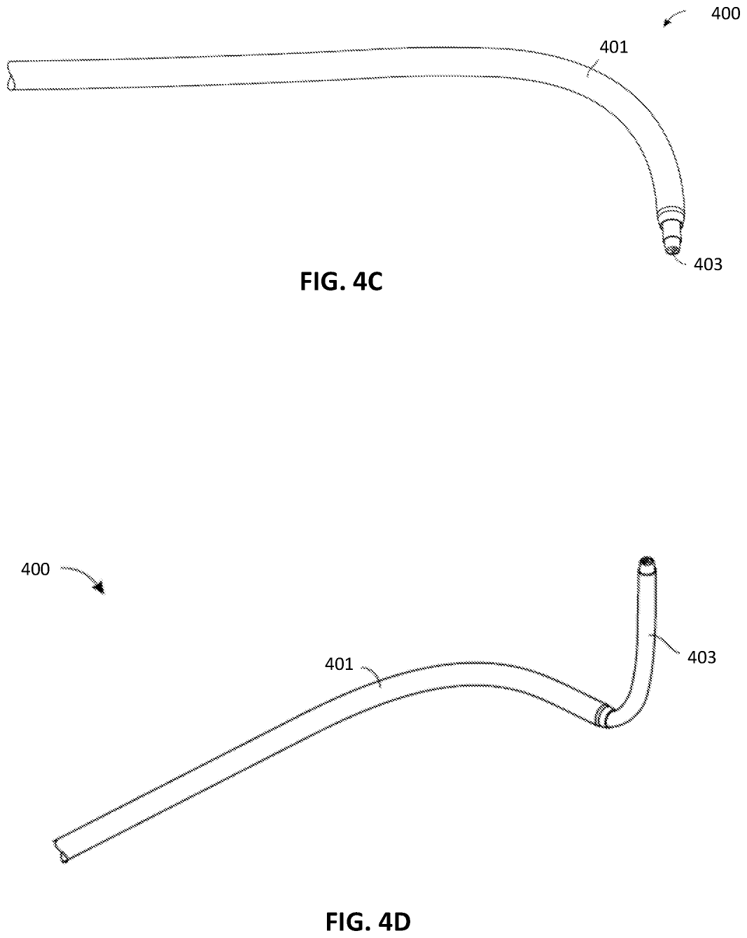

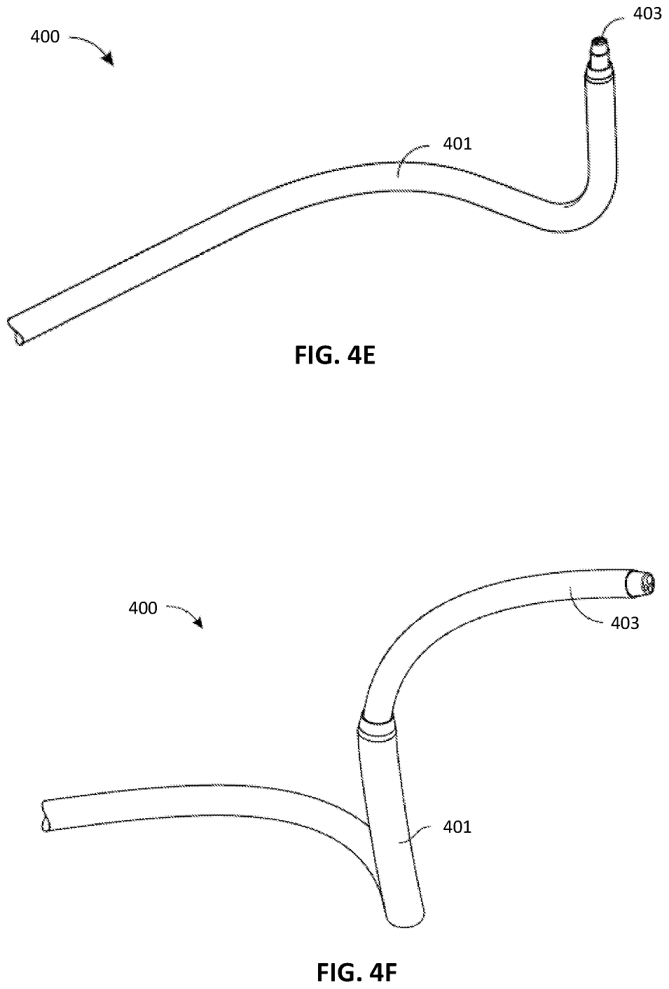

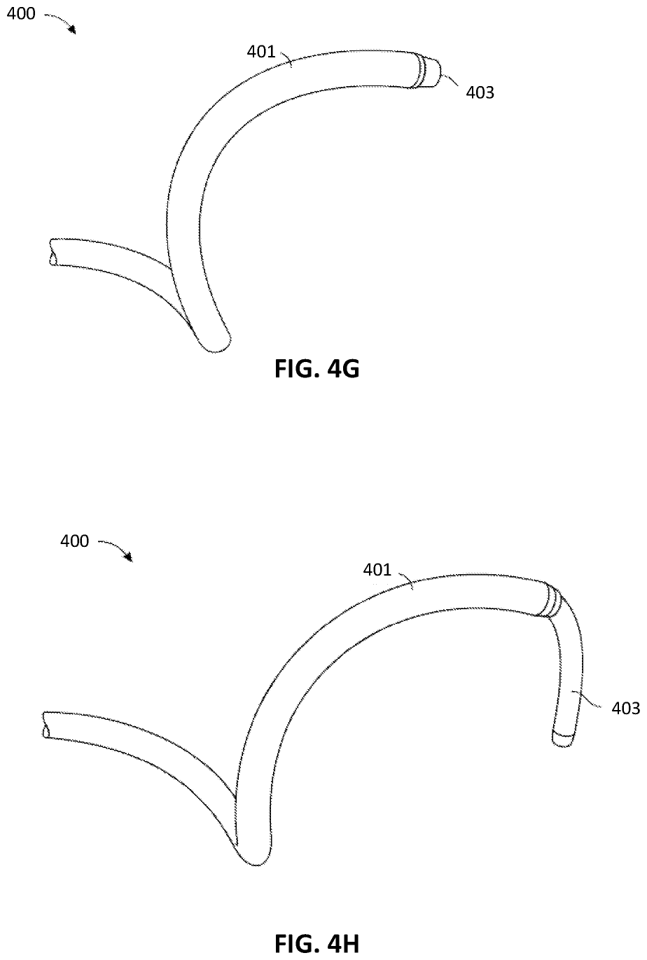

Further, any of the rigidizable devices described herein may be configured as nested apparatuses that may be nested to provide enhanced performance. For example, a nested apparatus (system) is shown in FIGS. 3 and illustrated in operation in FIGS. 4A-4H. In FIG. 3, the nested system 300 includes an outer rigidizing device 301 and an inner rigidizing device 302, configured as a rigidizing scope) that are axially and rotationally movable with respect to one another. In this example they move concentrically, but in some configurations they may be arranged non-concentrically. The outer rigidizing device 301 and the inner rigidizing device 302 can include any of the rigidizing features as described herein. For example, the outer rigidizing device 301 can include an outermost layer (e.g., tube) 305, a rigidizing (e.g., braided) layer 309, and an inner layer (e.g., tube) 315. Either or both the inner and outer tubes (layers) 305, 315 may be reinforced, e.g., including a coil wound therethrough. The outer rigidizing device 301 can be, for example, configured to receive vacuum between the outermost layer 305 and the inner layer 315 to provide rigidization. Similarly, the inner rigidizable device (scope 302) can include an outer layer 325 (e.g., with a coil wound therethrough in this example), a rigidizing (e.g., braid in this example) layer 329, a compression layer 321 (e.g., configured as a bladder layer in this example), and an inner layer 335 (e.g., with a coil wound therethrough in this example).

The inner rigidizing device (e.g., scope 302) can be, for example, configured to receive pressure between the compression layer 321 and the inner layer 335 to provide rigidization. Any of these rigidizing devices, including the inner rigidizing device shown in FIG. 3, may include an air/water channel 336 and a working channel 355 can extend with the inner rigidizing device 302. Additionally, any of these rigidizing devices (including the inner rigidizing device 302 shown in FIG. 3) can include a distal section 342 with a camera 365, lights 375, and steerable linkages 377. A cover 379 can extend over the end of the distal section 342. In another example, the camera and/or lighting can be delivered in a separate assembly (e.g., the camera and lighting can be bundled together in a catheter and delivered down the working channel and/or an additional working channel to the distal-most end). The feature of any of these apparatuses may include or be incorporated into apparatuses including flexible external working channels can be incorporated (as described in U.S. provisional patent application no. 13668-719.100). 63/342,618, titled “EXTERNAL WORKING CHANNELS FOR ENDOSCOPIC DEVICES,” and filed on May 16, 2022, herein incorporated by reference in its entirety.

The inner lumen 381 of the first, outer rigidizable device 301 can form a gap or interface 381 into which the second, inner, rigidizable device may be positioned. This gap or interface region 381 can have any appropriate dimensions, so that an annular space (d) remains around the second, inner, rigidizable device when inserted into the first, outer, rigidizable device. In some examples, when the inner rigidizable device is centered in the lumen of the outer rigidizable device, space on either side of the inner rigidizable device, d, may be between about 0.001″-0.050″, such as 0.0020″, 0.005″, or 0.020″ wide. The inner surface of the outer rigidizing device and/or the outer surface of the inner rigidizable device may be a low friction surface and may include, for example, powder, coatings (for example, hydrophilic or hydrophobic), or laminations to reduce the friction. In some examples, a seal may be present between the inner device 302 and the outer rigidizable device 301, and the intervening space can be pressurized, for example, with fluid or water, to create a hydrostatic bearing. In other examples, there can be seals between the inner rigidizable device 302 and outer rigidizable device 301, and the intervening space can be filled with small spheres to reduce friction.

The inner rigidizable device 302 and outer rigidizable device 301 can move relative to one another and alternately rigidize so as to transfer a bend or shape down the length of the nested system 300. For example, the inner device 302 can be inserted into a lumen and bent or steered into the desired shape. Pressure can be applied to the inner rigidizing device 302 to cause the rigidizing layer to rigidize the inner rigidizable device 302 in whatever configuration curve bend it had when the pressure was applied. The rigidizable device (for instance, in a flexible state) 301 can then be advanced over the rigid inner rigidizable device 302. When the outer rigidizable device 301 is sufficiently advanced relative to the inner rigidizable device 302, pressure (e.g., negative pressure in this example) can be applied to the outer rigidizable device 301 to cause the rigidizing layers to rigidize to fix the shape of the outer rigidizable device. The inner rigidizable device 302 can be transitioned to a flexible state, advanced, and the process repeated. Although the system 300 is described as including an inner rigidizable device configured as a scope, it should be understood that other configurations are possible. For example, the system might include two overtubes, two catheters, or a combination of overtube, catheter, and scope.

FIGS. 4A-4H illustrate the ability of these nested systems 400 to advance though very tortuous anatomy by control from the proximal end of the device while controlling the pressure (and therefore the rigidity/flexibility) in both the inner rigidizable device 403 and the outer rigidizable device 401. For example, FIG. 4A shows the nested system 400 inserted initially in a linear (straight) configuration. The distal end of the inner rigidizable device 403 may be steerable and may be extended from the outer rigidizable device 401 while being steered into a bend, as shown in FIG. 4B. The inner rigidizable device 403 may then be rigidized by the application of a pressure differential, and the outer rigidizable device 401 advanced distally over the locked curved shape of the rigid inner rigidizable device 403, as shown in FIG. 4C. Once at or near the distal end of the inner rigidizable device, the outer rigidizable device may be rigidized, (e.g., by a controller applying a pressure differential to the outer rigidizable device) and the inner rigidizable device may thereafter be transitioned into a flexible configuration. Thereafter, the inner rigidizable device 403 may then be advanced distally while being steered, using the rigid outer rigidizable device a stable platform to advance and steer, as shown in FIG. 4D. After being steered at least partially around the curve, the inner rigidizable device may again be rigidized and the outer rigidizable device may be made flexible and advanced distally over the rigid inner rigidizable device, as shown in FIG. 4E. The outer rigidizable device may then be made rigid (by applying a pressure differential) and the inner rigidizable device may be made flexible and advanced distally and steered, again, as shown in FIG. 4F; once the inner rigidizable device has been steered distally to the degree desired, the inner rigidizable device may be rigidized and the outer rigidizable device may be made flexible and advanced distally over the inner rigidizable device, as shown in FIG. 4G. Once the outer rigidizable device is advanced to the end of the inner rigidizable device it may be made rigid by applying a pressure differential, and the inner rigidizable device may be made flexible and steered while advancing distally, as shown in FIG. 4H. This process may be repeated as often as necessary to position the device, or any device associated with the device, in this shape-copying manner. The device may be retracted and/or the path corrected by reversing this process and withdrawing the outer rigidizable and inner rigidizable devices.

FIGS. 3 and 4A-4H are shown and described to illustrate generally the nested systems and methods of operating them that may be performed with any of the rigidizable devices described herein. With respect to FIGS. 4A-4H, the example nested apparatus shown may be rigidized by any appropriate method, including, but not limited to, the application of positive and/or negative pressure to one or both rigidizing members. The modifications to the rigidizing member, torsional stiffness, and/or durometer of the inner and/or outer layers (tubes) of these rigidizable devices may provide enhanced movement and functionality of the nested devices described herein when performing a method similar to that shown in FIGS. 4A-4H.

### Knit Rigidizing Layers

In any of the rigidizable devices described herein (and any nested systems or methods including them) may include a rigidizing layer formed of a knit material or knit layer (e.g., knit tube). The knit rigidizing layer, which may be referred to herein equivalently as a knit rigidizing layer or a knitted rigidizing layer, may be formed of a single fiber or may be knitted from multiple fibers. The fiber forming the knit may be a yarn, a filament, a mono-filament, a plurality of filaments, a strand, a thread, a wire, etc. The fiber may be made of a natural or synthetic material, including polymeric materials, metals and metal alloys, and a composite or a combinations thereof. In some cases the knit is formed of a polymeric material. The fiber may be continuous, in which each of the filament lengths forming the rigidizing layer are part of a single fiber, or they may be broken up into multiple filament lengths. For example, the knit material may be single fiber that is broken/cut at regular or irregular lengths.

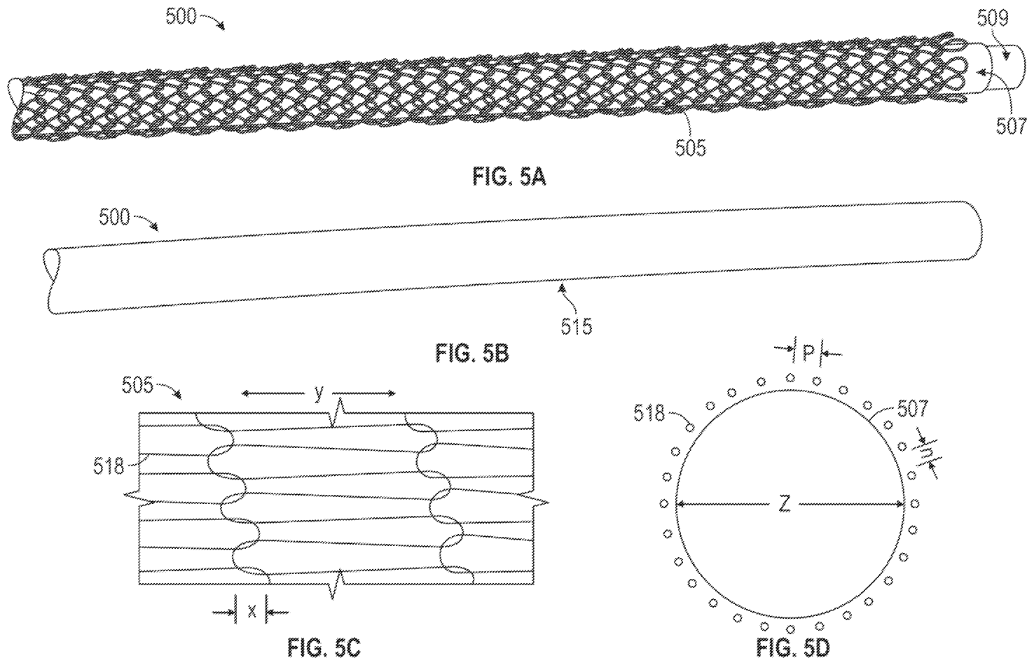

FIGS. 5A and 5B illustrate an example of a rigidizable device 500 including a knit rigidizing layer (e.g., tube) 505. In FIG. 5A the outermost layer (outer layer 515) is removed for clarity; FIG. 5B shows the rigidizable device with the outer layer 515 covering the other layers. This outer layer may be a reinforced outer layer, such as an outer coil-wound tube. In FIG. 5A the rigidizable device includes the knit rigidizing layer 505 extending over the elongate body of the device, including over a compression layer 507 (e.g., bladder) and an inner layer 509. The inner layer and the outer layer 515 may both be reinforced. This example, which is similar to the configuration shown in FIGS. 2A-2B (with the rigidizing layer 2109 configured as a knit layer 505), may be rigidized by the application of positive pressure between the compression layer 507 and the inner layer 509, which may drive the compression layer radially outward against the outer layer 515. Any of the other layers shown in FIGS. 2A-2B may be optionally included, including the gap regions/layer and the optional slip layer (which may not be necessary). This configuration may alternatively be actuated by the application of negative pressure, e.g., between the outer layer and the compression layer (including the region of the knit), which may draw the compression layer against the knit layer by the vacuum, rigidizing the layer.

A rigidizable device such as that shown in FIGS. 5A-5B may alternatively be configured so that positive pressure is applied between a compression layer (e.g., bladder) and the outer layer 515 (outer reinforced layer). In some examples the compression layer may be positioned between the outer layer and the knit rigidizing layer, so that positive pressure applied between the outer layer and the compression layer may rigidize the knit layer by driving the compression layer against the knit layer, into the inner (reinforced) layer. As in the configuration shown in FIGS. 5A-5B, the device may alternatively be actuated by the application of negative pressure, e.g., between the inner layer and the compression layer (including the region of the knit).

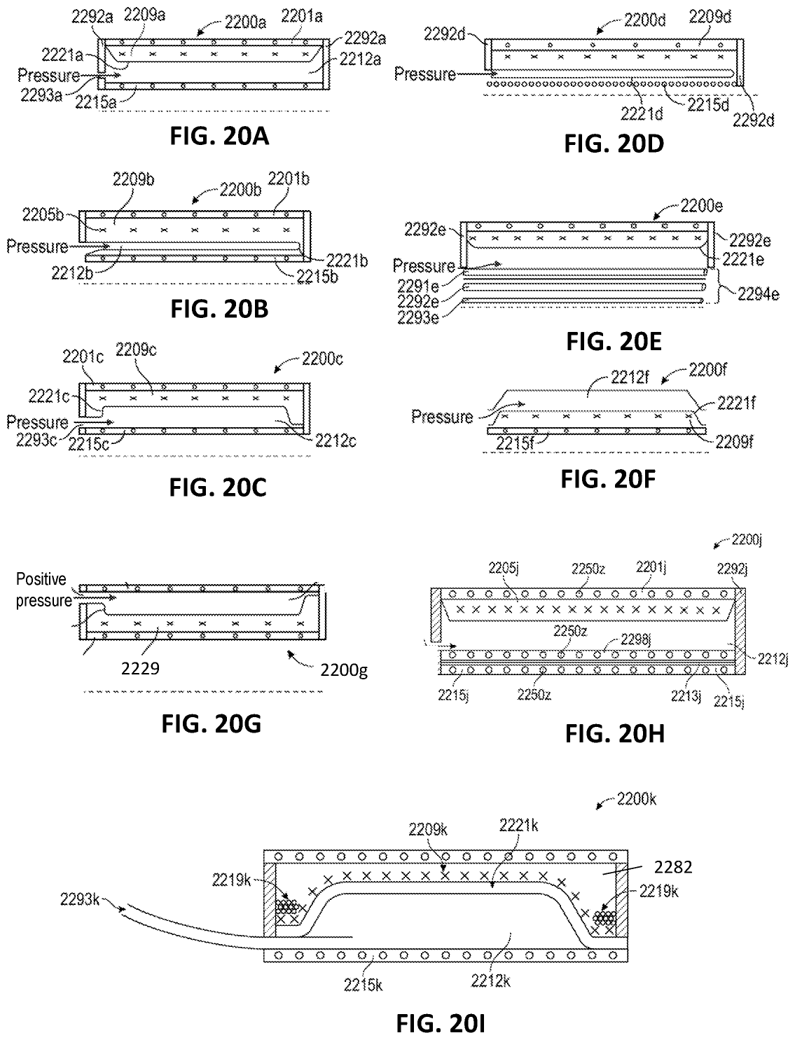

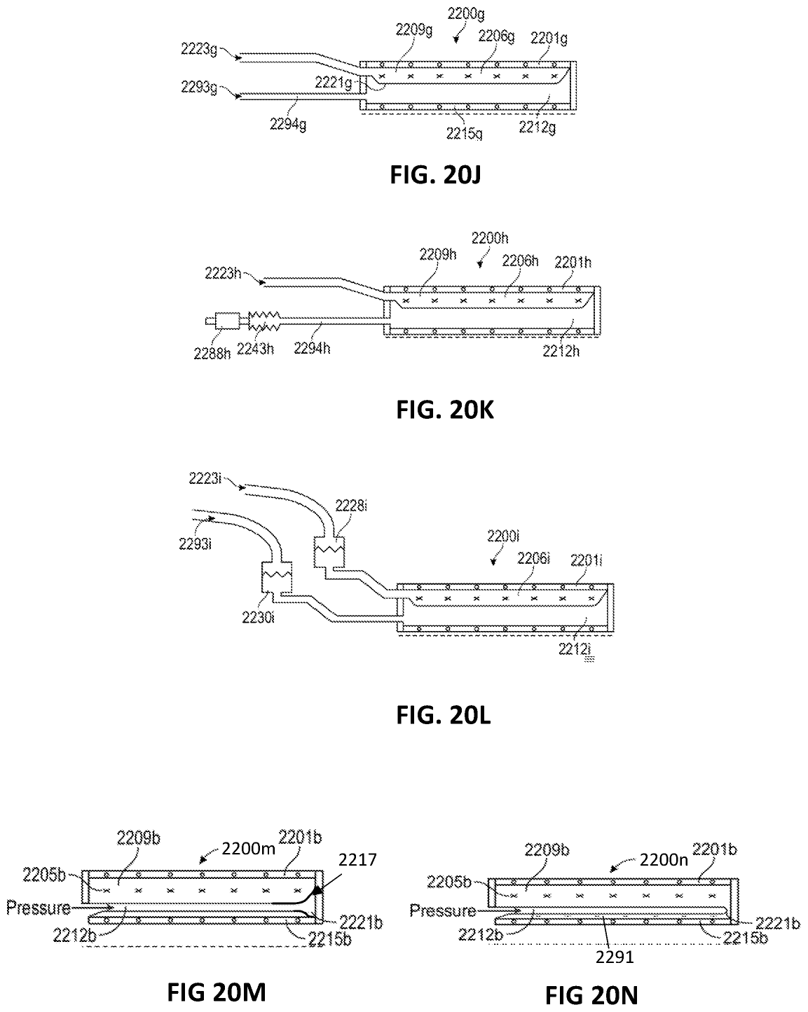

Alternatively, the rigidizable device including a knit rigidizing layer may be configured as shown in FIGS. 1A-1B and may be actuated by the application of negative pressure. In some examples the outer layer or the inner layer may be configured to as the compression layer (e.g., bladder) and may engage with the knit ridigizing layer when vacuum is applied. Examples of these alternative arrangements are described in FIGS. 20A-20N, below, and may include a knit rigidizing layer.

FIG. 5C illustrates one example of a portion of a knit layer 505 formed of a single filament 518 that forms interlocking loops. In the example shown in FIG. 5C the knit includes a plurality of stich loops each having a length, y, and a curved head and foot region having a length x. The stitch pattern shown in FIG. 5C is a weft knit pattern, but other knit patterns may be used. FIG. 5D shows an example of a transverse section through a knit layer positioned adjacent to a compression layer 507. In this example the knit layer is a tube having 28 strand segments that are formed of the same strand into loops (e.g., 14 loops that are arranged with the wale of the knit in parallel with the long axis of the device). The knit tube has a diameter, z, and the spacing between adjacent loops, n, is approximately equal around the circumference of the knit tube. The spacing between the stitch width, p, and the spacing, n, may vary along the length of the knit tube. The dimensions are illustrative only.

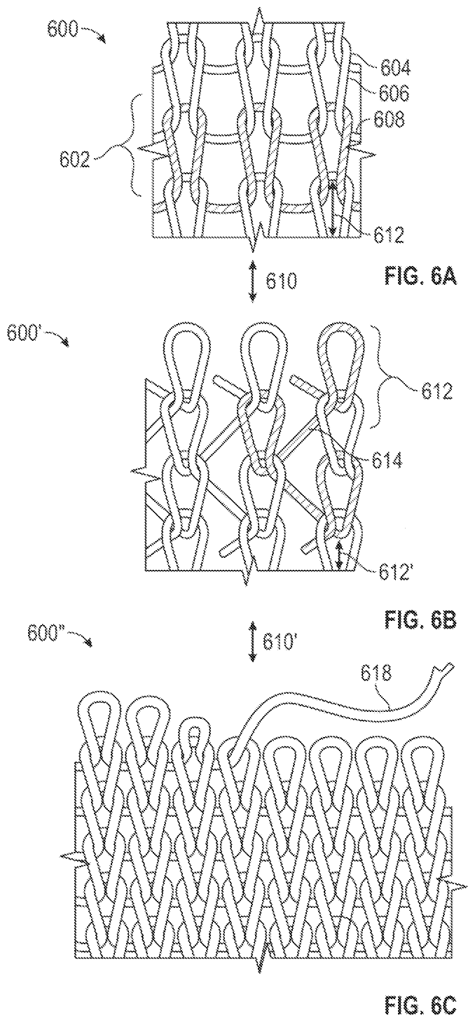

FIGS. 6A-6B illustrate two different examples of knits 600, 600′ that may be used. FIG. 6A shows a weft knit, similar to that shown in FIG. 5C. In this example the knit is formed of one or more strands (which may be continuous or broken/cut), forming stitch loops 602 that each include a head region 604, a pair of legs 606 and a first and second foot 608 where each foot engages with the head of a stitch loop in a course above or below the original stitch loop course. The connection between the feet of adjacent stitch loops may be referred to as the sinker (the sinker may also correspond to a head when the knit is rotate 180 degrees). In FIG. 6A the wale direct 612 extends up/down, and the course 610 extends right to left. Typically, a wale is a column of loops running lengthwise, corresponding to the warp of woven fabric in FIG. 6A. The course is a crosswise row of loops, corresponding to the filling of the resulting knit.

FIG. 6B illustrates an example of a warp knit 600′. In this example the warp knit also has a course 610′ and wale 612′ direction but the feet of each loop engage with the head region of a knit loop in a row (in the course direction) that is offset, as shown, forming a pattern of overlap 612 and underlap 614 lengths. The knit rigidizing layers described herein may use any appropriate pattern and may arrange the direction (course or wale direction) relative to the elongate axis (length) of the device. For example, the knit structure (the knit rigidizing layer) may be configured so that a wale direction of the knit extends in a long axis of the flexible tube. Alternatively, the knit structure may be configured so that a wale direction of the knit structure is perpendicular to a long axis of the flexible tube. Depending on the stitch length (y) relative to the loop diameter (p) and/or the spacing between loops (n), which may be related, it may be beneficial to arrange the knit rigidizing layer so that that either the wale or the course is arranged in parallel or perpendicular to the long axis of the elongate body of the rigidizable device. In any of the examples described herein, the knit structure may comprise an average loop length that is longer than the loop width. For example, the loop length may be two times or greater (e.g., 3×, 4×, 5×, 6×, 7×, 8×, 9×, 10×, 20×, 40×, 60×, 80×, 100× or more) than an average loop width.

Because knits (including knit tubes) may be stretched and compressed in bending without buckling or wrinkling, they may be particularly useful in the rigidizable devices described herein.

As shown in FIG. 6C a knit rigidizing layer 600″ may be formed of a single knitted fiber 618. As mentioned above the fiber may be formed of a single filament (monofilament) or a bundle of filaments (multi-filament). The pattern shown therefore includes a plurality of lengths of filaments (e.g., an array of filament lengths) that cross each other in the knit pattern. In FIG. 6C the plurality of lengths of filaments that cross over and under each other are all part of the same fiber or strand. In some examples the knitted fiber or strand may be cut or divided into multiple separate filament lengths. The knit material (e.g., the fiber) may be formed of any appropriate material, such as a metal, metal alloy, polymeric material, natural fiber, etc.

### Woven and Braided Rigidizing Layers

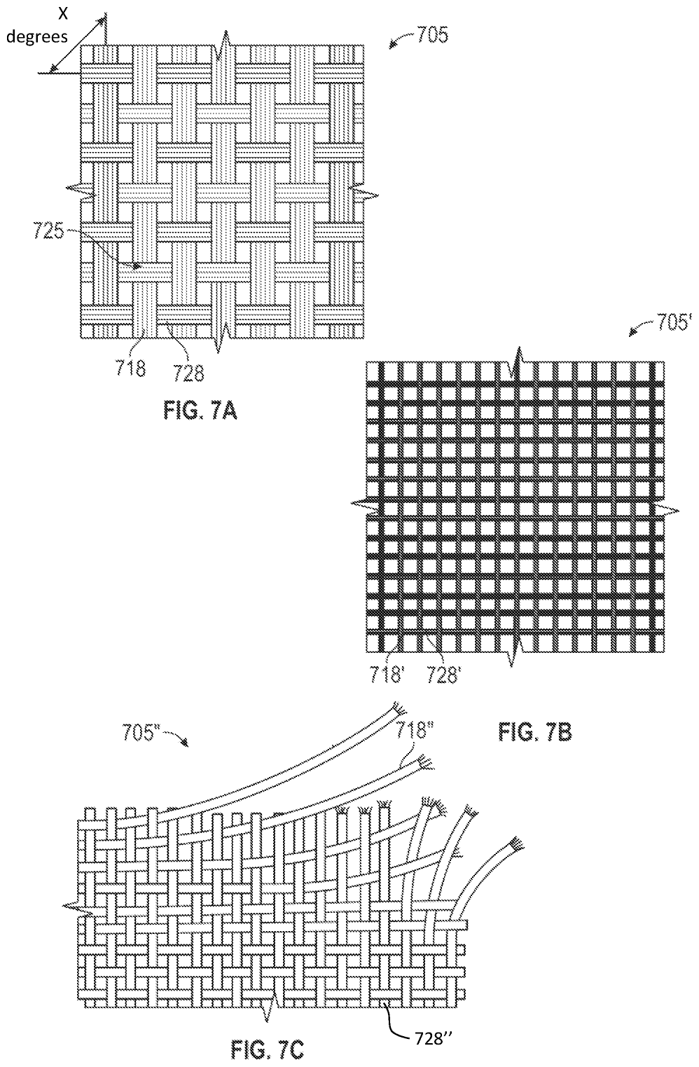

In any of the rigidizable devices described herein (and any nested systems or methods including them) may include a rigidizing layer that is woven. FIGS. 7A-7C illustrate an example of a woven a rigidizing layer 705 that may be used as the rigidizing layer of the rigidizable device and may be arranged as shown in FIGS. 1A-1B, 2A-2B or 20A-20N. In FIG. 7A the weave includes a plurality of parallel fibers that form a set of intersecting fibers; in FIG. 7A the fibers intersect with each other at 90 degree angles, but this angle may vary (e.g., between about 30 degrees and 150 degrees, 45 degrees and 135 degrees, 50 degrees and 130 degrees, 70 degrees and 110 degrees, 80 degrees and 100 degrees, etc.). The pattern of intersecting filament lengths (e.g., the array of filament lengths) includes individual filament lengths that cross over and under each other as shown; a first filament length 718 crosses over a second filament length 728 and under a third filament length 725. In this example, the pattern shown in an under-over pattern, but this pattern may be different for other examples of rigidizing layers; in FIG. 7A the pattern is one over, one under. In some examples the pattern may be two over two under, or two over and one under, etc. Any appropriate fiber (e.g., strand) may be used to form the rigidizing layer, a mentioned for knit rigidizing layers above. In the woven rigidizing layer shown in FIG. 7A the fiber is a muti-filament fiber including a bundle of multiple filaments forming each strand. FIG. 7B shows an example of a woven rigidizing layer 705′ formed of a monofilament, also arranged with parallel strands 718′, 728′ arranged in a woven pattern similar to that shown in FIG. 7A. The woven pattern may be any desired tightness (e.g., pore size). In general, as shown in FIG. 7C, multiple different lengths of fibers 718″, 728″ are used to form the woven pattern 705″.

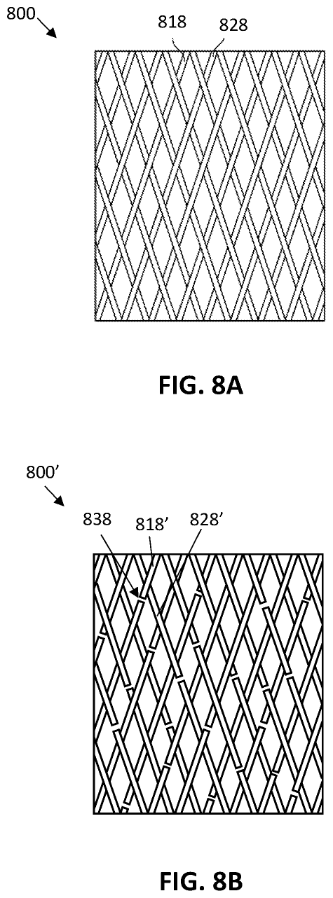

FIGS. 8A and 8B illustrate examples of braided rigidizing layers. In FIG. 8A. In FIG. 8A the braid 800 is formed of a plurality of fibers 818, 828 that are arranged in an over-and-under pattern having a braid angle relative to the long axis (e.g., the long axis of the device when included as the rigidizing layer). In general, the braid angle (relative to the centerline along the central axis) of the braided rigidizing layer (tube) may be 45 degrees or less (e.g. less than 45 degrees, 40 degrees or less, less than 40 degrees, 35 degrees or less, less than 35 degrees, 30 degrees or less, 20 degrees or less, less than 20 degrees, etc. In FIG. 8A the different filaments forming the braid layer are continuous and unbroken. However in some examples it may be beneficial to include breaks or cuts, as illustrated in FIG. 8B. In this example, the material includes a plurality of breaks or cuts 838 in the braided strands. Although such an arrangement may be undesirable in a fabric or even in a braid used as part of a medical device, this disrupted (e.g., broken or cut) arrangement may be beneficial in the context of a rigidizing layer. Thus, in

FIG. 8B the braided pattern 800′ forming the rigidizing layer (e.g., rigidizing tube) may enhance flexibility in the un-rigidized configuration, while permitting a high degree of rigidizing in the actuated state. Thus, in FIG. 8 the strands 818, 828′ cross over and under each other in the braid pattern shown but are cut 838 periodically along their lengths. The number or density of the cuts may be varied; in some examples the fibers may be cut after every crossing over or under another fiber, while in other examples the fibers may be cut after every 2 (or 3, or 4, or 5, or more) crossings. The cut pattern may be non-uniform. In some examples it may be beneficial to have the cuts or breaks distributed at a density of between about one cut/break for every third crossing, etc. (e.g., between every second and every 25^{th} crossing, every third and every 20^{th} crossing, etc.).

Other rigidizing layers (e.g., knit, woven, etc.) may also include breaks or cuts. These breaks or cuts may be formed during fabrication by laser cutting, mechanical cutting, or any other appropriate cutting technique.

### Pressure-driven Rigidization

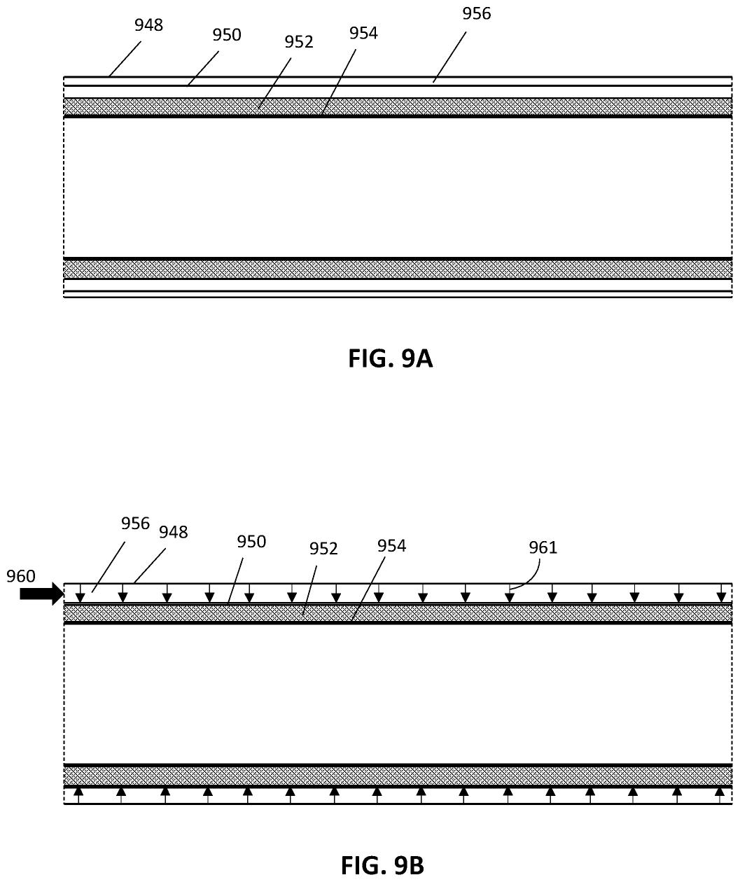

As mentioned above, in general, these apparatuses may be configured to be rigidized by the application of pressure. This is illustrated schematically in FIGS. 9A-9B for a generic rigidizing layer. In this example the device is shown in longitudinal cross-section through a portion of the length of the device. The layers forming the device are arranged as concentric tubes. In FIG. 9A the device is shown without the application of pressure, and includes an inner layer (tube) 954, an outer layer (tube) 948 and a compression layer (e.g., bladder) 950 and a rigidizing layer 952. The particular configuration shown illustrates the rigidizing layer 952 between the inner layer 954 and the compression layer 950. A first gap layer 956 is present between the outer layer 948 and the rigidizing layer 952. A port (not shown) may be present at an end (e.g., a proximal end region) of the device to couple to the source of pressure (e.g., positive pressure). A second gap layer may be present between the compression layer 950 and the rigidizing layer 952, and/or between the rigidizing layer 952 and the inner layer 954. In the configuration shown in FIG. 9A the device may be flexible as each of these layers may slide relative to each other when bending the device. In particular, the rigidizing layer may flex and slide relative to the inner layer 954 and the compression layer 950.

FIG. 9B illustrates the device of FIG. 9A when positive pressure 960 is applied between the outer layer 948 and the compression layer 950. Alternatively the compression layer may be a bladder into which the positive pressure is applied. In FIG. 9B, as positive pressure is applied the compression layer 950 is driven 961 against the rigidizing layer 952, so that is compressed between the compression layer 950 and the inner layer 954 (and/or any intervening layers). Compressing the rigidizing layer 952 rigidizes the device. Any bends or curves are preserved without changing the shape.

In the example shown in FIGS. 9A and 9B, any appropriate rigidizing layer 952 may be used, including knit compression layers, woven, braided, granules, scales, etc.

In some examples, particularly those having elastic (e.g., elastomeric) compression layers and rigidizing layers formed of filament lengths that cross over and under each other, the compression layer may deform into the rigidizing layer, which may enhance the rigidity of the device. For example, as pressure is applied, the compression layer (e.g., bladder) may apply force directly to the rigidizing layer. Depending on the bladder type, the bladder may deform, depress, or interdigitate into the space around and between the elements (e.g., filaments, wires, etc.) of the rigidizing layer. Conforming to the overlapping (over-and-under) fiber or filament lengths may help lock the rigidizing layer relative to the inner layer (or in some examples outer layer) to which it is being compressed. The application of positive pressure in this manner may therefore increase rigidization as positive pressure is increased even beyond what is otherwise expected. Thus a rigidizing layer comprising a plurality of filament lengths crossing over and under each may be generally configured so that, in the flexible configuration, the filament (e.g., fiber) lengths may shear relative to each other. However, when positive pressure is applied, the deformable compression layer may be pushed against the rigidizing layer so that the compression layer may conform to or deform into or between the plurality of filament lengths to prevent shear of the plurality of filament lengths relative to each other.

### Combined Rigidizing/Compression Layers

In some examples the rigidizable devices described herein may include a combined or hybrid rigidizing layer and compression layer. Rather than the discrete rigidizing layers described above, in some cases the rigidizing layer may be integrated (e.g., encapsulated, laminated within, etc.) a deformable compression layer. This configuration may also be referred to as a rigidizing layer, but it may be described as an array of filament lengths encapsulated within a compression material. The array of filament lengths may be configured to slide over each other (e.g., shear) when the compression material is in a first, uncompressed, configuration, and array of filament lengths may be engaged against each other when the compression material is in a second, compressed, configuration, preventing them from sliding. In this example, the filaments may be partially encapsuled within the compression material; in some cases within a channel or passage through the compression material for individual or groups (e.g., at crossing regions) of fibers. Deforming the compression material, by the application of pressure, may result in increasing the shear force on the filaments, particularly where two or more filaments overlap each other.

This configuration may work with either negative pressure (vacuum) or positive pressure. For example, positive pressure may be applied from the outside of the rigidizing layer (driving the compression material against the fibers (filaments) encapsulated therein. Negative pressure may be applied within the compression material, e.g., within the channels holding the filaments (fibers), collapsing the channels into/on the filaments.

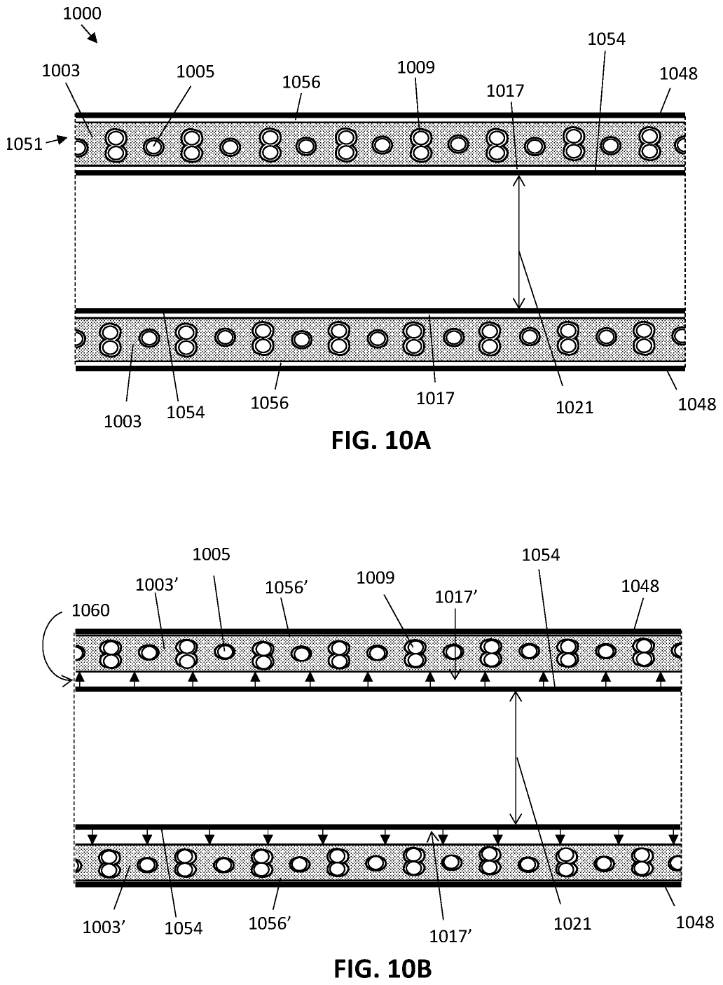

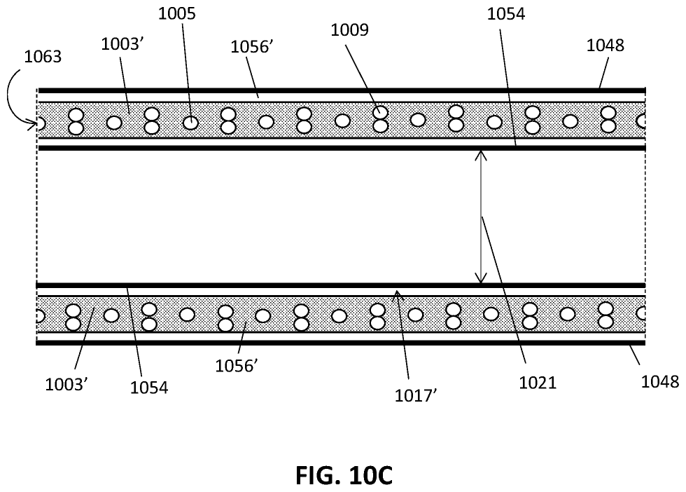

FIGS. 10A-10B illustrate examples of a rigidizable device 1000 including a combined rigidizing and compression layer 1051 in which the filament lengths 1005 are loosely encapsulated within a compression material 1003. The lengths of filaments may be continuous or discrete but include regions where two or more lengths cross over each other and may shear 1009 within the compression material. In some examples it may be preferrable to have the filament lengths be discrete and relatively short (e.g., between 0.2 cm and 10 cm, between 0.5 cm and 10 cm, between 0.5 cm and 7 cm, etc.). The filament lengths may cross over each other in a pattern, such as a woven or mesh pattern, or in a random pattern. In FIG. 10A the device is shown in a flexible state, in which the filaments may move freely within the compression material. The filaments may be monofilaments or multi-filaments and may be formed of a polymeric material, a metal or metal alloy (e.g., wire), or any other appropriate material or combination of these. As shown, the rigidizable device 1000 also includes an outer layer (tube 1048) formed of an elongate flexible tube, and an inner layer (tube 1054) also formed of an elongate flexible tube. Either or both the inner and outer layers may be reinforced. The device includes an inner lumen 1021. In any of the devices described herein the device may be configured so that there is minimal (e.g., less than 5%) or no change in the inner and/or outer diameter even under the application of relatively high pressure or vacuum. The rigidizing layer 1051 (including the loosely encapsulated lengths of filament) may be positioned between the outer and inner layers and may be separated by a gap, such as a first gap 1056 and a second gap 1017.

FIG. 10B illustrates and example of a device that is actuated by the application of positive pressure 1060 in the second gap, e.g., between the inner layer 1054 and the rigidizable layer 1051, resulting in compressing the rigidizable layer 1051 against the outer layer 1048, as shown. Within the rigidizable layer 1051 the compression causes the compression layer to deform, drastically increasing the shear forces on the filaments within the loosely encapsulated channels holding the filaments. The rigidizable layer 1051 may be thin and configured to compress under the positive pressure. In FIG. 10B, the compression of the rigidizable layer 1051 along its length may therefore rigidize the rigidizable layer 1051 by preventing shear of filament strands.

Alternatively, in some examples positive pressure may be applied to the first gap region 1056 between the outer layer (tube 1048) and the rigidizable layer 1051 (not shown), driving the compression layer against the inner layer 1054.

FIG. 10C illustrates an example of rigidization of the rigidizable device of FIG. 10C by the application of negative pressure within the rigidizable layer 1051, e.g. within the channels in which the filaments are loosely encapsulated. Applying vacuum within these channels may collapse the channels against the filaments in a manner that is analogous to the application of external positive pressure. In some examples positive pressure may be applied within the channels in order to make the device more flexible and the device may be rigidized by removing the positive pressure and/or applying negative pressure to further rigidize.

### Rigidizing Layers having Sub-Layers (e.g., fingers, scales and/or plates)

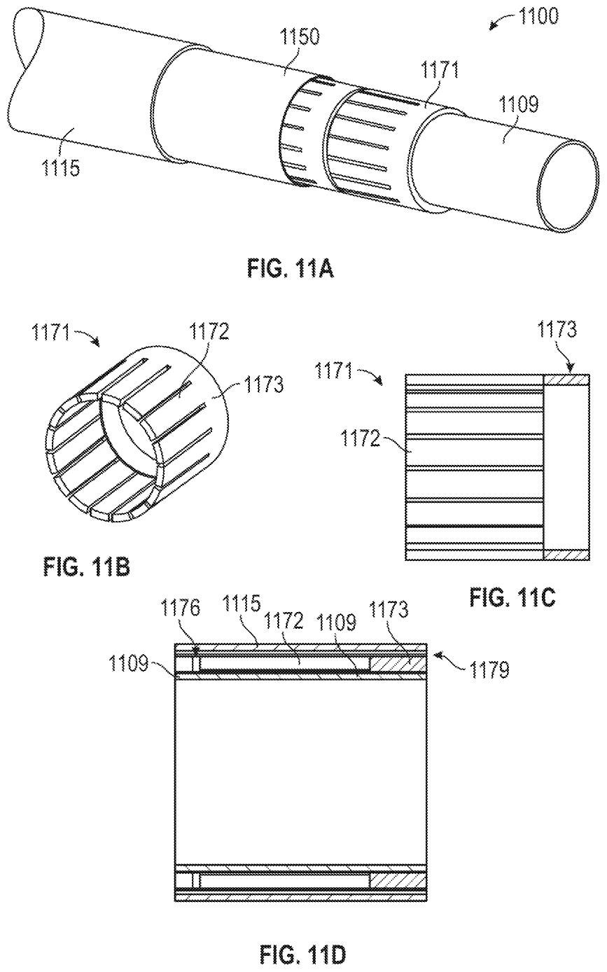

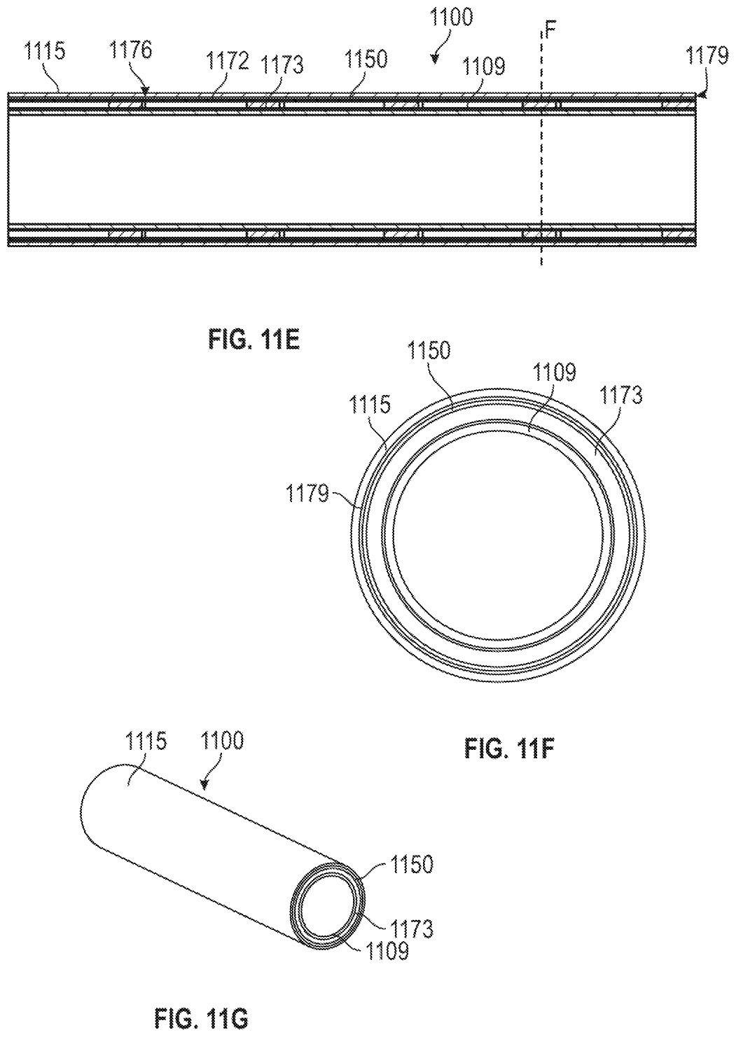

In some examples the rigidizable devices may include a rigidizing layer having sub-layers that may be configured as fingers, scales and/or plates. The sub-layers may be driven by the application of pressure (e.g., positive pressure), against either other fingers, scales and/or plates, or against another layer of the device (e.g., the inner layer or outer layer) in order to rigidize them. Shapes may be flat or rounded. Their surfaces may be textured, surface modified, grabby, have features to transfer shear loads, or have frictionally engineered surfaces. These sub-layers may otherwise be flexible in bending yet have high axial stiffness. For example, FIGS. 11A-11G illustrate an example of a device including a plurality of segments 1171 each including a plurality of radially arranged arms or fingers 1172 that extend from an attachment region 1173. These sub-layers may overlap with other arms or not, and the application of positive pressure (e.g., from a compression layer) may force the sub-layers against another layer, rigidizing the device. For example, FIG. 11A shows a section through an example of a rigidizable device 1100 having an outer layer (tube) 1115 that is reinforced, a compression layer 1150, an inner layer 1109 that may also be reinforced, and a rigidizing layer comprising a plurality of axially arranged segments 1171.

FIGS. 11B and 11C show isolated views of a segment. In this example the segment includes a plurality of arms 1172 extending from a ring-shaped attachment section 1173. The segment may be made of a material (metal, polymer (mylar, PEEK, PEN), composite, etc.) that may be deflectable so that the arms may be deflected, and preferably elastically deflected, radially inward or outward, e.g., by the compression layer (e.g., bladder 1150) when driven by positive pressure. FIG. 11D shows a section through a portion of the device 1100 of FIG. 11A. in FIG. 11D the segment(s) 1171 are positioned between the compression layer 1150 (e.g., bladder) and an inner layer 1109. An air gap 1179 that may be fluidly connected to an inlet to couple to a source of positive pressure (not shown) is positioned between the outer layer 1115 and the compression layer 1150. In this example the segments are non-overlapping, at least in the unbent configuration shown. Thus, a series of segments 1172 may be arranged along the length of the device in a spaced configuration, as shown in FIG. 11E. In some examples the device may include spaces or gaps between the segments 1172 that are sufficiently large so as to prevent overlap between them as the device is bent.

FIG. 11F shows a radial section through the rigidizable device 1100 of FIGS. 11A-11E, at line F in FIG. 11E. The outer layer (tube) 1115 may be a reinforced, e.g., outer coil-wound tube, and the inner layer (tube) 1109 may also or alternatively be reinforced, e.g., as an inner coil-wound tube. The compression layer 1150 is between the attachment section of a segment 1173. FIG. 11G shows a perspective view of a portion of the rigidizable device shown in FIG. 11E and 11F. When pressure is applied through an inlet in fluid communication with the gap region, the compression layer (e.g., bladder) pushes the segment arms 1272 against the inner tube (e.g., the inner coil-wound tube, ICWT), thereby rigidizing the device. The greater the pressure applied, the greater the more rigid the device may become. This rigidization is achieved by jamming the sub-layers (e.g., arms, scales, etc.) against an adjacent layer, such as the inner layer (inner tube), or the outer layer.

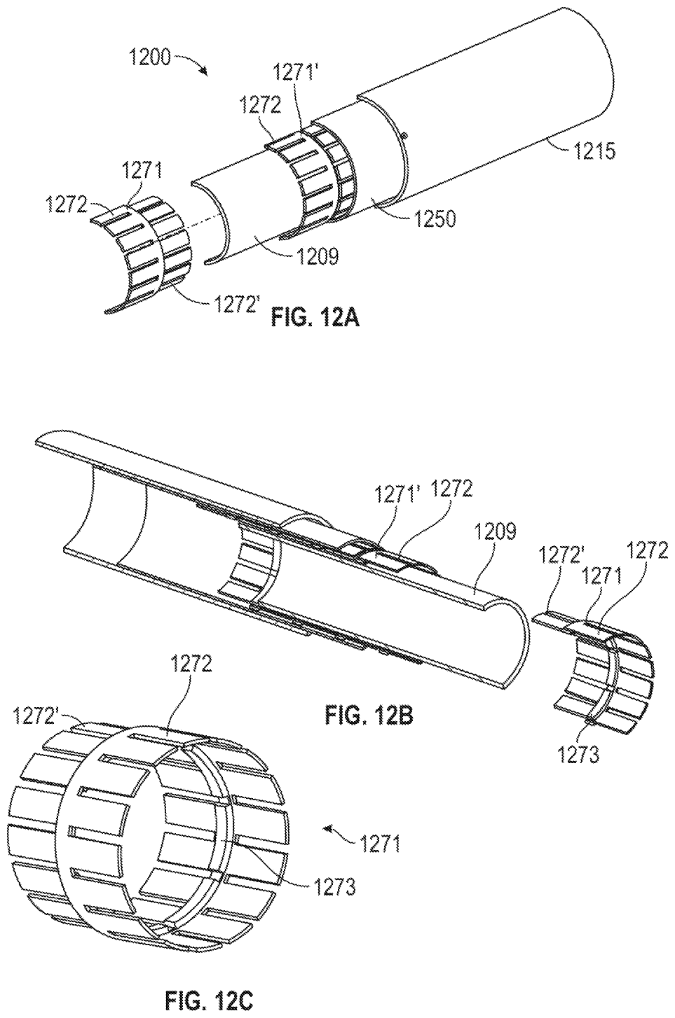

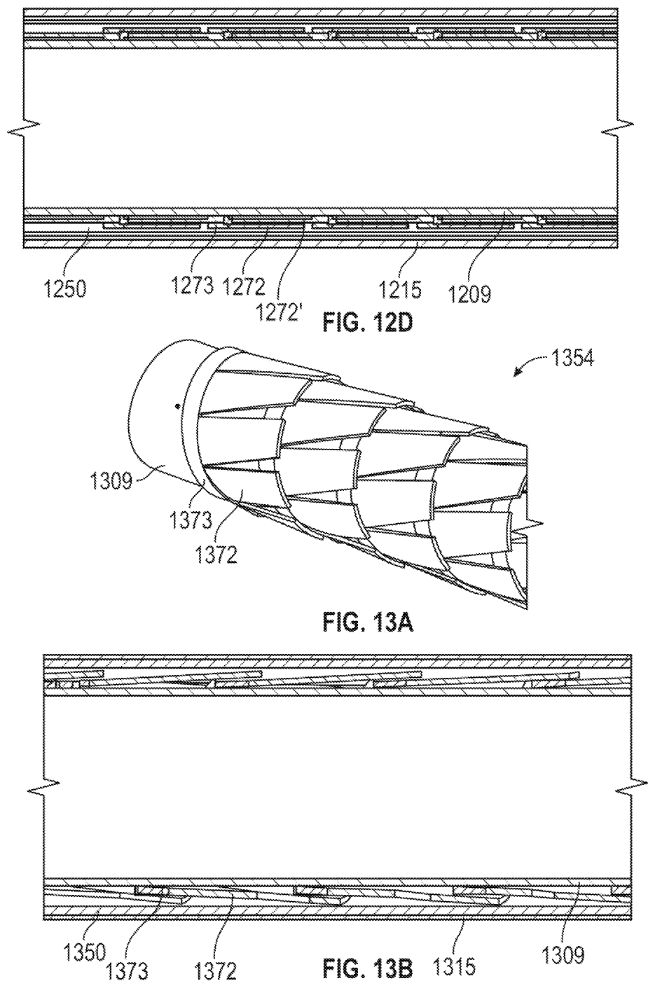

In some examples the longitudinally adjacent members (e.g., segments) are configured to overlap with each other, in addition to being adjacently arranged along the length of the device. For example, FIG. 12A shows a portion of a rigidizable device 1200 in a first exploded view, and FIG. 12B show an alternative exploded view of the same rigidizable device. In this example the device, including a plurality of adjacent and overlapping members (e.g., segments in this example) having a plurality of scales, plates or arms 1272, 1272′ extending from a central annular attachment segment 1273. The attachment segment 1273 may extend fully or partially around the radius of the device. In this example, in contrast to the segment shown in

FIGS. 11A-11G, arms 1272, 1272′ extend both distally and proximally from the attachment section 1273. The device also includes an outer layer (e.g., an outer coil-wound tube, OCWT) 1215, and an inner layer (e.g., an inner coil-wound tube, ICWT) 1209, and a compression layer (e.g., bladder) 1250.

In FIGS. 12A-12D, the segments 1271 are configured to overlap with each other so that the arms 1272, 1272′ are radially overlapping. As shown in FIG. 12C the segment is configured so that the arms extending distally will fit over the arms extending proximally from an adjacent segment. As in FIGS. 11A-11G the segment may be made of any appropriate material, particularly those materials that can be elastically deformed when compressed against each other to rigidize the device. For example, the segments may be formed of a polymeric material or a metal or metal alloy. In this configuration the material has low bending stiffness but high axial stiffness.

FIG. 12D illustrates a section through an assembled rigidizable device as shown in FIGS. 12A-12C. In the un-rigidized (flexible) configuration the device may bend relatively freely, and the adjacent and overlapping sub-layers (e.g., arms, scales, plates, etc.) may freely slide over each other. However, the application of positive pressure to drive the compression layer against the rigidizing layer formed of the plurality of segments including deflectable sub-layers (e.g., arms, scales, plates, etc.) may rigidize the device in whatever bend or curve it was in when positive pressure was first applied; the greater the positive pressure differential, the greater the rigidity. In this example, the plates are not able to readily slide against each other or against the inner layer.

Another example of a rigidizing layer formed of a plurality of sub-layers (e.g., or as in FIGS. 11A-11G and FIGS., 12A-12D, a plurality of segments including sub-layers) is shown in FIGS. 13A-13B. In this example, rather than annular rings of segments having sub-layers extending from an attachment region, the rigidizing layer is a helically arranged or spiral wrapped plurality of sub-layers (e.g., arms, scales, plates, etc.) that overlap each other. FIG. 13A shows an example of a portion of a rigidizable device showing an arranged plurality of sub-layers 1372 (e.g., curved plates) that wind around an inner layer (tube) 1309 and overlap with adjacent plates. The sub-layers 1372 are shown extending from a helically wound attachment region 1373; one or more attachment regions may be used. FIG. 13B shows a portion of a rigidizable device 1300 that includes the rigidizing layer 1354 of FIG. 13A. The device also includes an inner layer 1309 that may be reinforced, and outer layer 1315, a compression layer 1350 (e.g., bladder), and a helically-wound rigidizing layer 1354 comprising a plurality of sub-layers 1342 extending from an attachment region 1373. The application of positive pressure drives the sub-layers against each other and against the inner layer (in this example) and rigidizes the device.

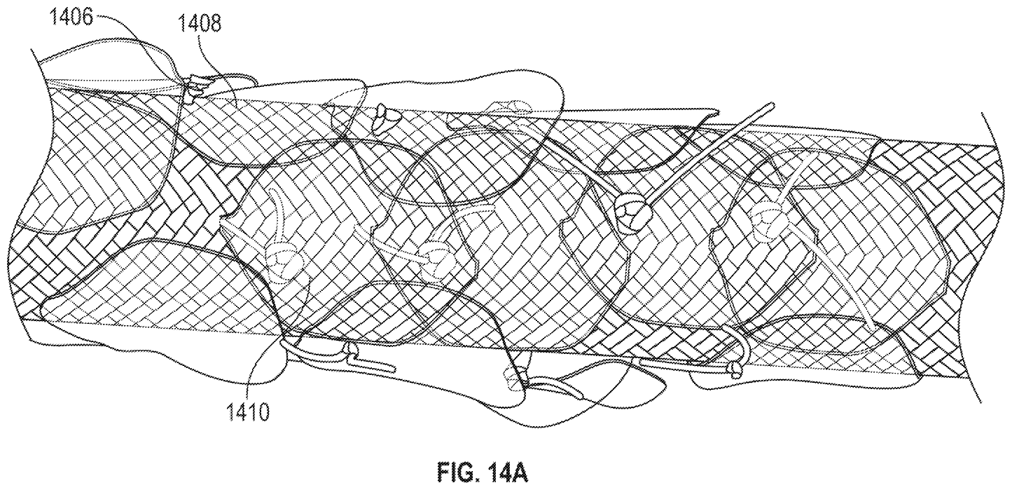

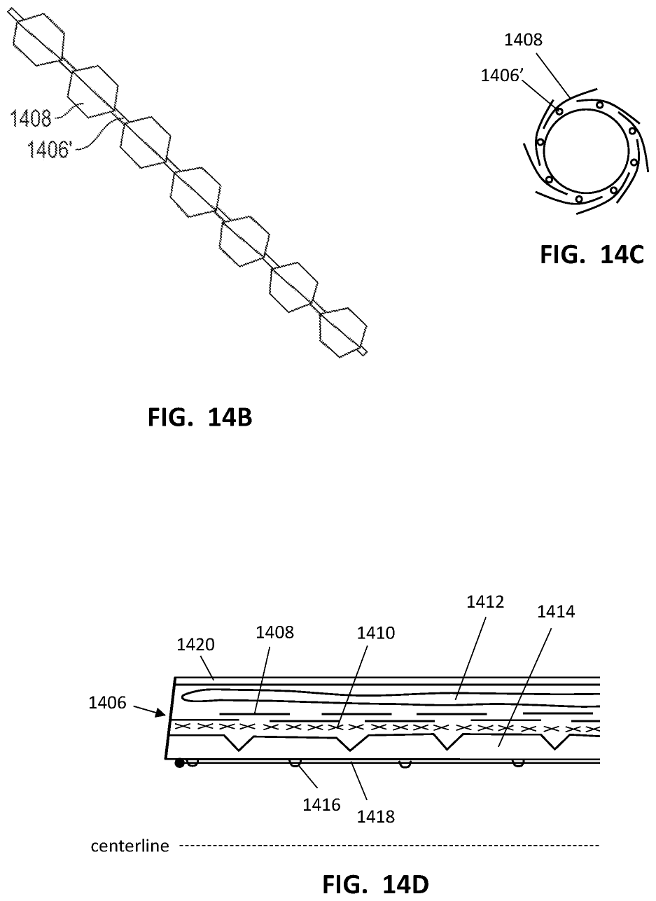

FIGS. 14A-14D illustrate another example of an apparatus (e.g., device) including a rigidizing layer formed of a plurality of sub-layers, shown in this example as scales 1408. As shown in FIG. 14A, scales can be attached as individual elements, e.g., attached to the inner layer (tube) 1410. As described above, the scales can be configured such that each one slides over the other, or they may be spaced apart and non-overlapping. In FIG. 14A the scales are attached to the inner layer 1410 by an attachment 1406 (e.g., tie, adhesive, weld, etc.) that may flexibly attach the scale. In FIG. 14A, the scales forming the rigidizing layer are attached to an underlying structure (a braid) of the inner layer 1410 by a tied suture. Alternatively, the sub-layers (scales) may be attached to a central ‘backbone’ 1406′ (for example, a wire, or base material connecting the scales), as shown in FIG. 14B. This backbone 1406′ may be attached at one end and may slide at another as the structure is bent or articulated, in which case the inner or outer path length may change. FIG. 14C shows a section through the rigidizing layer formed by the plurality of overlapping scales 1408 and the inner layer 1410.

FIG. 14D schematically illustrates a partial longitudinal section through one portion of a wall of an example of a rigidizable apparatus in which the rigidizing layer is formed of a plurality of overlapping sub-layers (e.g., scales). In this example, an outer layer (e.g., an OCWT) 1420 is radially outward (from the centerline of the device) of a compression layer (e.g., bladder 1412) and the compression layer is between the rigidizing layer 1408 and the outer layer. The scales 1408 of the rigidizing layer may be attached or may simply overlay the inner layer (e.g., an ICWT) 1410; the inner layer includes a braid. In this example, the portion of the apparatus shown is taken from the distal tip region 1406 and also shows a steering assembly including a plurality of bendable (e.g., hinged) linkages 1414) and a cable 1418 (held to the linkages by one or more slidable attachments 1416) that may be pulled to bend and steer the distal tip. Any of the apparatuses described herein may include a similar arrangement of linkages and tendons (e.g., cables) for steering. In this example, the linkages and tendons may be arranged radially inward from the inner layer (tube), and may be, for example, within the inner lumen of the device. Alternatively, the steerable assembly (e.g., linkages and tendon/cables) may be integrated into apparatus radially outward of inner layer.

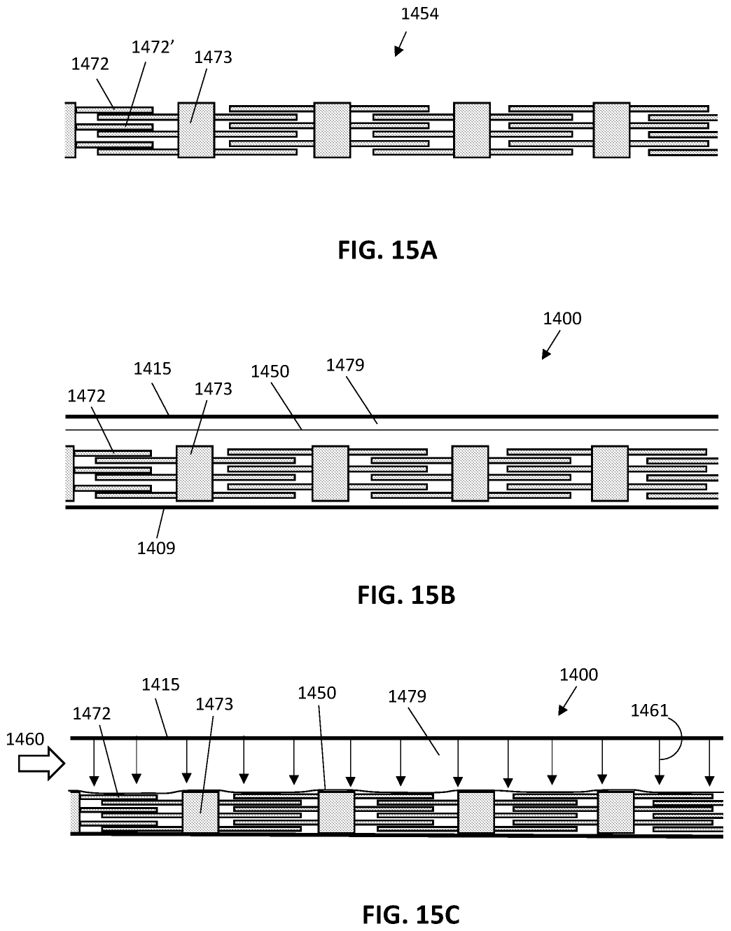

FIG. 15A-15C illustrates another example in which a plurality of overlapping arms may extend radially in parallel and engage with adjacent arms. In FIG. 15A the rigidizing layer 1454 comprising a plurality of sub-layers 1472, 1472′ extending from an attachment region 1473. The sub-layers may be configured as arms, plates, scales, etc., and may freely slide relative to each other when pressure is not applied against them. The rigidizing layer may include multiple attachment regions (e.g., as multiple different segments, similar to that shown in FIGS.

11A-11G and 12A-12D) or as a single helically-wound attachment region, similar to that shown in FIGS. 13A-13B. FIGS. 15B and 15C illustrate and example of a device 1400 that includes a rigidizing layer 1454 similar to that shown in FIG. 15A. The device may also include an outer layer 1415, an inner layer 1409 and a compression layer 1450. FIG. 15B show the device without a pressure differential; the device may be highly flexible, as the inner layer, outer layer and rigidizing layer are all very flexible. FIG. 15C shows the same device with positive pressure 1460 applied between the outer layer 1415 and the compression layer 1450, e.g., within the gap region 1479, driving 1461 the compression layer against the rigidizing layer so that the arms 1472 of the rigidizing layer compress against each other, rigidizing the device. The greater the pressure applied, the greater the rigidity.

### Rigidizing Layers having Granules/Particles

In some examples the rigidizing layer of a rigidizable device may include granules (e.g., particles) that may be converted, by the application of pressure and in particular by the application of positive pressure, from a loose and flexible configuration into a packed and rigid configuration that are ‘jammed’ together. In some examples the granules may be held within container (bag, cylinder, etc.) that is permeable to air and is flexible and compressible, and a separate compression layer (e.g., bladder) may be driven against the granules (e.g., against the container) to compress them, allowing air to escape from the container, but the container serving to more effectively retain the media. Alternatively in some examples the compression layer may form part of the container.

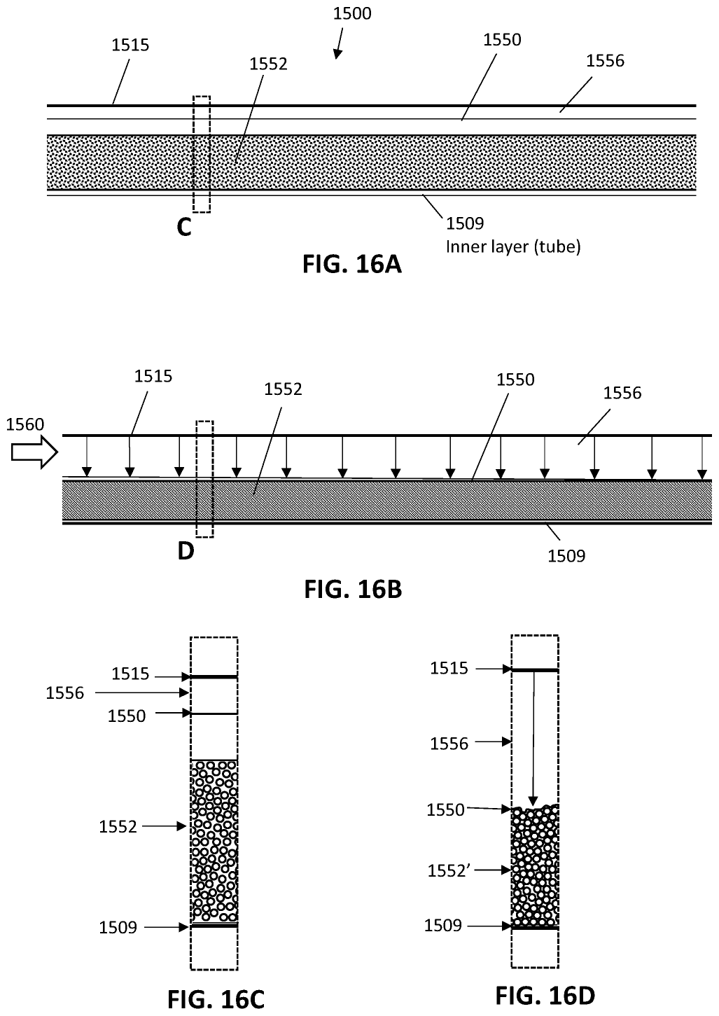

For example, FIGS. 16A-16D illustrate an example of a rigidizable device 1500 including a rigidizing layer formed of particles 1552 that can be rigidized by the application of positive pressure. The device includes an outer layer (e.g., tube) 1515, and an inner layer (e.g., tube) 1509, and a compression layer 1550 that is, in this example, between the outer layer and the rigidizing layer. Alternatively, the compression layer may be between the inner layer and the rigidizing layer. An air gap is between the compression layer and the outer layer; a second air gap may be between the compression layer and the rigidizing layer, and a third gap may be between the rigidizing layer and the inner layer. Thus all of these layers may slide in the flexible configuration allowing bending and flexing of the device. The particles or granules within the rigidizing layer may be loosely packed, so that the rigidizing layer may be highly flexible in the unpressurized configuration. The device may also include a release pathway (e.g., for releasing air from the rigidizing layer (e.g., between the granules) when compression is applied by the compression layer. For example FIG. 16B illustrates rigidization of the device 1500 of FIG. 16A. In this example, positive pressure 1560 is applied into the gap 1556 and/or into the bladder (of the compression layer 1550) so that the compression layer is driven against the rigidizing layer, as shown. The granules or particles of the rigidizing layer are packed, preventing down against the inner layer 1509, making the device rigid without significantly changing the profile of the device.

FIGS. 16C and 16D show enlarged sections of FIG. 16A (flexible configuration) and FIG. 16B (rigidi configuration), respectively. As shown in FIG. 16C, the rigidizing layer 1552 includes a plurality of loosely-packed granules or particles. In FIG. 16D the compression layer 1550 drives packing of the granules or particles of the rigidizing layer 1552′, as the rigidizing layer is compressed between the compression layer and the inner layer in this example. The amount of packing and the rigidity achieved may depend on the pressure applied and in some examples on the shape and/or sizes of the particles or granules. In general, the granules may be any appropriate material, typically a rigid or semi-rigid material (e.g., polymer, metal, etc.). That is either uncompressible or elastically compressible. In some cases, to prevent against harm to the patient should the device rupture, the granules may be formed of biocompatible and/or bioresorbable material. The granules may have any appropriate shape; for example, the granules may be round, square, faceted (e.g., crystalline), etc. In some examples the shape of the granules may be uniform or non-uniform. The granules may include both uniform and non-uniform shapes and may include a variety of different shapes and/or sizes in the same rigidizing layer.

### Torsional Stiffening Members

There are multiple clinical advantages to creating a close relationship between a proximal input rotation and a resultant distal output rotation. Ideally this ratio would be one-to-one (for example, 360 degrees of input rotation yielding a commensurate 360 degrees of output rotation). This benefit is magnified in mother-child (e.g., nested, or endoscope-overtube) systems, because a mother device may provide a rigidized tube through which a child can rotate about its inner central axis. This allows very precise central-axis oriented rotation in distal anatomy, something that cannot be achieved by an endoscope alone. This allows the user to orient a camera or a tool and allows for a tool that grips on local tissue and can then is manipulated precisely. This is of value to both manual systems and to robotic systems. With robotic systems, for example, the user could precisely titrate rotation, down to very small increments (e.g., one degree or even less), merely by pushing a button on a control device. This degree of control may be powerful and has not previously been achievable.

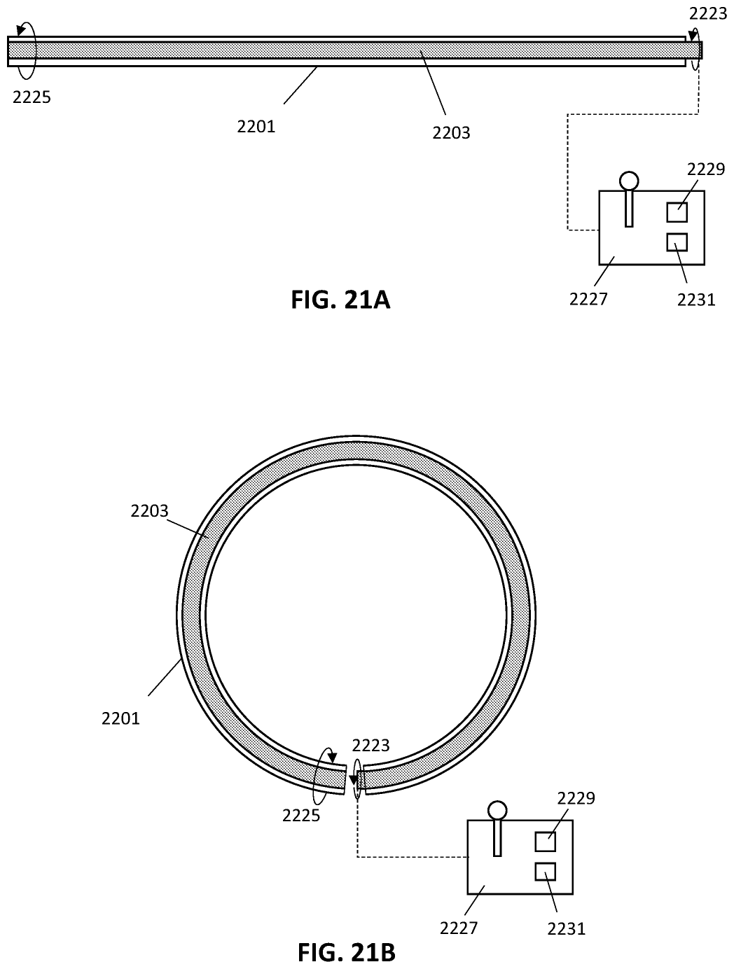

For example, for a 360 degree rotational input, the rotational output could be 360 degrees, 350 degrees, 340 degrees, 330 degrees, 320 degrees, 310 degrees, 300 degrees, 290 degrees, 280 degrees, 270 degrees, 260 degrees, 250 degrees, 240 degrees, between about 180 and 240 degrees, etc. This may be clinically meaningful and may be performed with a certain amount of tortuosity, for example, the data can be normalized to a situation in which the devices are turned through 360 degrees of curvature. FIGS. 21A and 21B illustrates an example of a section through a system including a torsionally stiffening device. In FIG. 21A the device includes an outer rigidizing device (mother) 2201 and an inner rigidizing device (child) 2203. The device is shown in a straight configuration. Torquing (e.g., rotating or twisting) the inner rigidizing device 2223 in a first direction by any selected angle (e.g., 1 degree, 2 degrees, 3 degrees, 4 degrees, 5 degrees, 10 degrees, 15 degrees, etc.) results in rotation of the distal end region of the inner rigidizing device 2225 by the same selected angle. In general, torque from the proximal end of the inner rigidizing device may be faithfully transmitted to the distal end of the inner rigidizing member even with the inner rigidizing member in the flexible configuration. The outer rigidizing device may be rigidized when torquing the (flexible) inner rigidizing device.

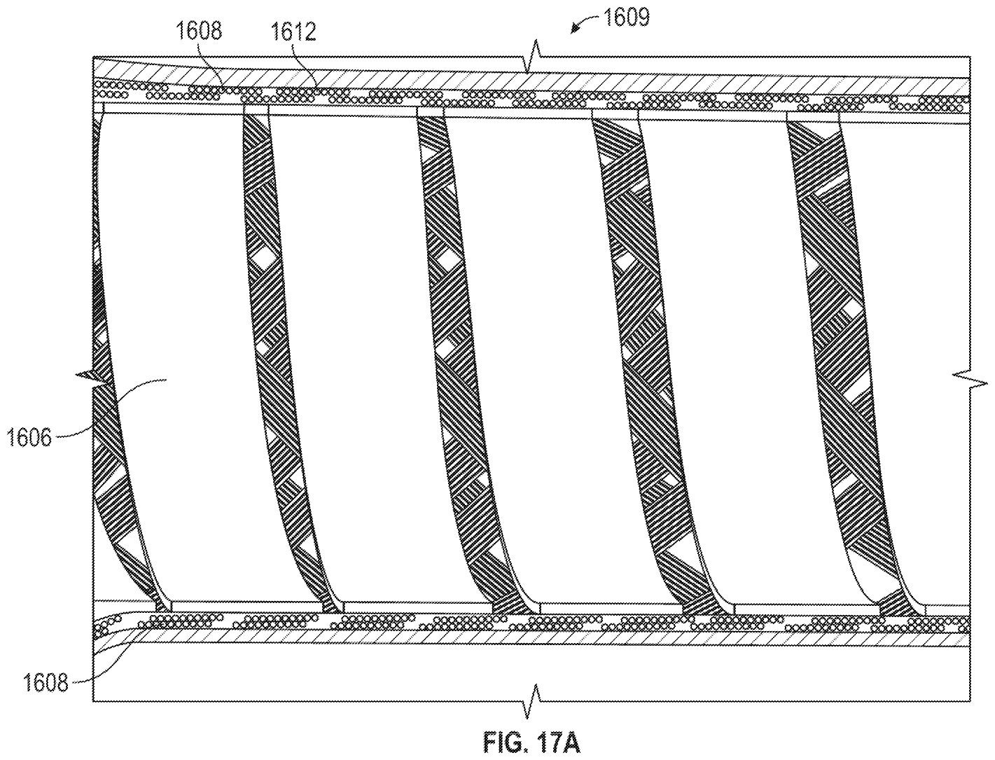

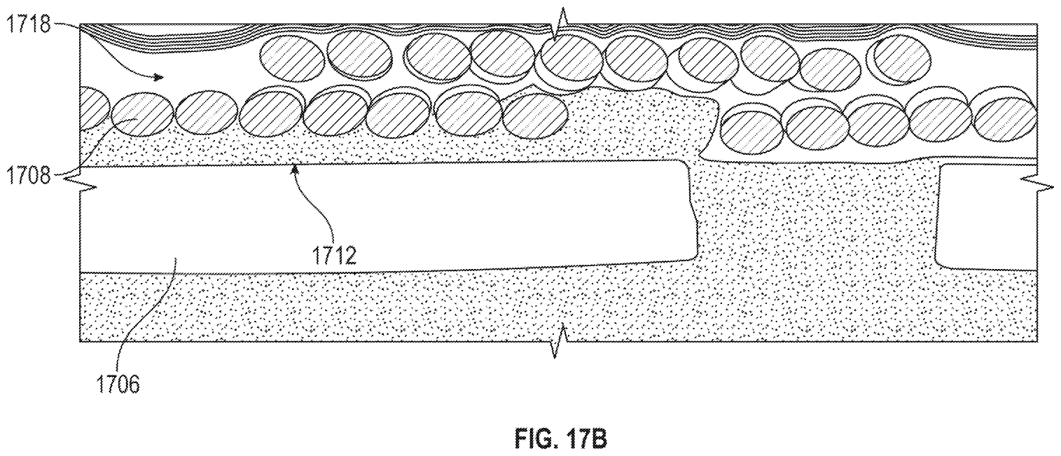

Significantly, the apparatuses described herein may also faithfully transmit torque down the full length of the inner rigidizing device even when the apparatus is curved in any arbitrary curve path, as shown in FIG. 21B. In FIG. 21B the outer device (or mother) 2201 has a circular shape (i.e., shown in this example as 360 degrees of curvature), with the inner device (or child) 2203 inserted inside. With the mother is rigidized, the child is torqued while in the flexible configuration; for a given input torque (rotational input) 2223, the rotational output of the child is measured. In the examples described herein, the apparatus is configured so that the output torque is approximately the same as the input torque. For a given input rotation an output rotation of approximately the same (e.g., input rotation +/− a lag margin) may result, even with the device in a highly curved configuration. The lag margin may be relatively low (e.g., 15% or less, 14% or less, 13% or less, 12% or less, 11% or less, 10% or less, 9% or less, 8% or less, 7% or less, 6% or less, 5% or less, 4% or less, 3% or less, 2% or less, etc.). For example, for an input rotation of 360 degrees, an output rotation of 330 degrees may be achieved if the lag margin is approximately 8.3%. The apparatuses described herein including a torsional stiffening layer (as described above in reference to FIGS. 17A-17B may be configured so that the lag margin is relatively low. Any of these apparatuses may also be configured to reduce drag between the inner rigidizing device 2003 and the outer ridigizing device 2001. For example, a lubricious material may be used. In some examples the outer surface of the inner rigidizing device and/or the inner surface of the outer ridigizing device may be lubricated and/or may be formed of a lubricous material. The lubricous material may be a hydrophobic material or a hydrophilic material. In the example shown in FIG. 21B, the flexible inner ridigizing device may be driven against the inner wall of the rigid outer rigidizing device as the inner device is torqued. Although FIGS. 21A-21B illustrate torquing of the inner rigidizing device relative to the outer rigidizing device, any of these apparatuses may also be configured so that torque is transmitted faithfully when the outer rigidizing device (e.g., including a torsional stiffening layer) is rotated relative to the inner rigidizing device. For example, the outer rigidizing device may be torqued in the flexible configuration when the inner rigidizing device is rigidized so that the outer rigidizing device faithfully transmits torque along the length of the device, e.g., with high fidelity (e.g., very low lag margin).

In general, torquing (e.g., rotation) of the apparatus, e.g., the inner apparatus and/or the outer apparatus, may be performed manually, automatically (e.g., robotically) or semi-automatically. For example, a robotic system may include one or more motors driving rotation. In FIG. 21A and 21B, the example includes a user input device 2227 that includes one or more inputs for controlling movement (rotation, and in some examples other steering control). For example, as shown schematically in FIGS. 21A and 21B, the user input device 2227 may include control inputs, such as a clockwise rotation button, counterclockwise rotation button, etc. These controls may be processed through software, algorithms, and/or actuators.

Any of the apparatuses described herein may include torsional stiffening elements. In particular, the high-pressure devices described herein may include a torsional stiffening layer. The torsional stiffening layer may be integrated into the inner and/or outer layers. Alternatively, it may be free-floating such that is not intentionally attached to adjacent layers. For example, a rigidizing device may include: a flexible inner tube that is configured to provide torsional stiffening. The flexible inner tube may comprise a first coil (e.g. of wire, ribbon, etc.). It may include a secondary or additional coils. It may include a torsional braid and a flexible material at least partially surrounding or between the coil and the torsional braid. The flexible material may form the body of the tube. A device including a flexible inner tube with a torsional stiffening layer may also include a flexible outer tube, a rigidizing layer between the inner and outer tubes, an inlet configured to attach to a source of positive pressure and a compression layer. The flexible material may serve as one of the leak-proof members for rigidization. Alternatively, it may provide structure for the inner coil-wound tube (ICWT) but may not explicitly be leak-proof. For example, as shown in FIG. 20B, both leak-proof pressurization member(s) may be a bladder. This bladder 221b could be ‘out and back’, as shown in FIG. 20B (i.e., attached at only one end), or it may be two distinct tubes that are bonded at either end 2217, as shown in the rigidizing device 2200m of FIG. 20M. In FIG. 20N the rigidizing device 2200n is similar to rigidizing device 2200b and includes a pressure gap 2212b is surrounded by a compression layer comprising an everted bladder layer 2221b (or a double-layered bladder), i.e., such that the bladder layer 2221b includes one side that borders the rigidizing layer 2205b and one side that borders an innermost layer 2215b that including a second rigidizing layer 2291. As pressure is supplied to the pressure gap 2212b (inside of the two sides of the bladder layer 2221b), the bladder layer 2221b can expand both against the innermost layer 2215b and inner rigidizing layer 2291 and against the rigidizing layer 2209b (which in turn can be pushed against the outermost layer 2201b).

In general, the torsional stiffening layer may be integrated into a flexible tube such as the inner and/or outer layers (tubes) in any of the apparatuses (devices and systems) described herein. For example, FIG. 17A illustrates an example of a tubular structure (such as an inner coil-wound tube) configured to include a torsional stiffening layer. FIG. 17A shows a section through the tube, which is configured as a flexible inner coil-wound tube 1609. In this example the flexible tube includes a first coil wire 1606 shown as a flat wire (or ribbon) that is helically wound around the tube on the exposed inner surface. Radially outward from the flat wire is a torsional stiffening braid 1608 that is attached at discrete locations (attachment points) 1612 to the outer surface of the flat wire coils 1606. A flexible material (e.g., a polymeric coating or laminated elastomer) may be applied over the torsional stiffening braid and/or between the torsional stiffening braid and the flat wire. The flat wire is left exposed in this example but may be covered by a flexible polymeric material.

The torsional braid may comprise a plurality of filaments. The torsional braid may have a braid angle of greater than about 40 degrees (e.g., 40 degrees or more, 45 degrees or more, 50 degrees or more, 55 degrees or more, etc.) relative to the long axis of the device. The torsional braid may be formed of a polymeric or metallic material.

In any of these apparatuses, the first coil wire and the torsional braid may be coupled to each other at discrete regions along the length of the flexible inner tube. For example, the first coil wire and the torsional braid may be coupled to each other between every 30-720 degrees of the helically wound coil wire (e.g., between every 60-720 degrees, between every 90-720 degrees, between every 180-720 degrees, between every 270-720 degrees, between every 1-2 turns, between every 1-5 turns, etc.).

In any of these examples, the first coil wire and/or the torsional braid may be at least partially encapsulated within a material (e.g., an elastomeric material). For example, the torsional braid may be at least partially encapsuled in a polymeric material. FIG. 17B illustrates another example of a section through an inner coil-wound tube (ICWT) having a torsional stiffening layer. In the example the tube includes a flat wire coil 1706 that may be helically wound as described in FIG. 17A; the flat wire coil can be one or more, e.g., two, coils). The flat wire coil 1706 is coupled to a torsional braid (torsional stiffening braid) 1708 at discrete attachment points 1712 on the outer side of the flat wire coil 1706. As mentioned above, the torsional braid may include multiple filaments per bobbin so that they may be applied around the tube at a relatively high coverage ratio. In FIG. 17B, ten parallel braid wires are shown. In FIG. 17B the device also includes an encapsulating material 1718; the encapsulating material may be on or around (at least partially around) the torsional braid. In some examples the encapsulating material may provide a full pressure seal around the tube; in other examples the tube (e.g., the inner tube or outer tube) including this material is not sealed. The torsional braid may be directly coupled to the flat wire coil, or it may be coupled via the one or more attachment points 1712. In some examples the torsional braid is coupled to the flat wire coil through the encapsulating material. This may keep the coil in place and prevent it from floating adjacent to, but not linked to, the flat wire coil and other portions of the apparatus.

The modified inner and/or outer elongate flexible tube may also include a compression layer (e.g., bladder) as described above. In some examples the encapsulating material may provide a seal for use or as part of the compression layer. In some cases less (or no) encapsulating may be used.

The same or a similar torsional stiffening layer may be incorporated into an outer layer (e.g., as an outer coil-wound tube) in any of the devices described herein.

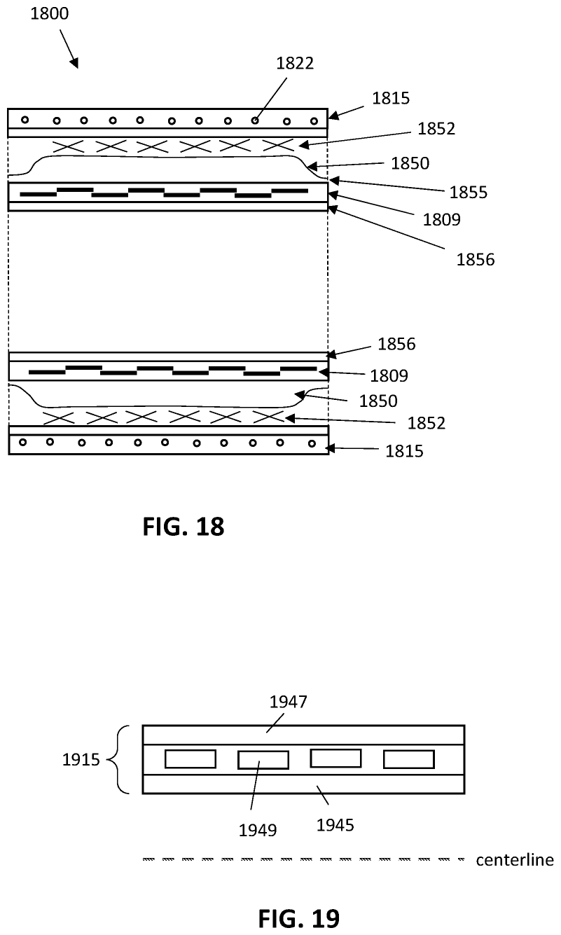

FIG. 18 schematically illustrates an example of a section through a device including an inner coil-wound tube as described above. In this example, the device 1800 includes a reinforced outer layer 1815, which is reinforced with a plurality of filaments 1822 wound around the layer (or within the layer). The inner tube (e.g., inner coil-wound tube, ICWT) 1809 may include one or more reinforcing wires, as shown schematically in FIG. 18. The inner coil-wound tube 1809 may be adjacent to an inner layer or coating 1856, such as a lubricous coating or the like. A compression layer (e.g., bladder layer) 1850 may be positioned adjacent to the inner coil wound tube 1809, and a rigidizing layer (or any of the rigidizing layers described herein) may be used and positioned so that he application of positive pressure to the bladder, or between the outer tube and bladder. FIG. 18 also shows a pressure inlet 1855 into the bladder 1850 to couple to a source of positive pressure (not shown).

A device such as the one shown in FIG. 18 may therefore be rotated about its long axis and the torsional rigidity provided by the torsional layer incorporated into the ICWT may allow it to track rotation of the proximal end with rotation of the distal end. In some examples the outer tube (OCWT) 1815 may also or alternatively be configured to include a torsional stiffening layer.

For nested systems, this configuration could be incorporated into the mother, the child (daughter), both, or neither the mother nor the child.

### Inner and/or Outer Layers having Different Durometers

Any of the inner or outer layers (tubes) described herein may be configured so that one side of the tube has a higher (harder) durometer than the opposite side. This could be valuable for both high pressure (positive pressure) systems and vacuum systems, as well as for both ICWTs and OCWTs. For example, for a positive pressure system, an outer coil-wound tube (OCTW) may include an outer portion of the tube (radially more distant from the tube's centerline) that has a durometer that is higher than a durometer of the inner portion (radially less distant) of the tube. Surprisingly, the inventors have found that this configuration may result in significant performance improvements. For example, the outside surface exhibits enhanced abrasion resistance, and the inner surface more optimally interfaces with the rigidization layer, resulting in enhanced rigidization values. For example, a device may include an inner elongate tube and an outer elongate tube, in which the outer elongate tube includes an inner region, a reinforcing member, and an outer region, wherein the inner region has a durometer lower than the durometer of the outer region. The outer region may have a durometer of between about 70 Shore A and about 80 Shore D, whereas the inner region may have a durometer of between 30 and 90 Shore A on the Shore A scale. The inner layer (tube) may also include different durometer regions but the arrangement relative to the inner region and the outer region may be reversed as compared to the outer layer. For example, for the ICWT, the inner elongate tube may include an inner region, a reinforcing member, and an outer region, wherein the outer region (radially more distant) has a durometer lower than the durometer of the inner region (radially less distant). For example, the outer region may have a durometer of between about 30 and about 90 Shore A on the Shore A scale, whereas the inner region may have a durometer of between about 70 Shore A and 80 Shore D. This configuration achieves the goals of a tougher surface facing outward (for example, for improved performance relative to devices that slide through its inner diameter), while maintaining a softer surface facing inward so as to improve rigidization, should the rigidization layer be pushed against this surface.

FIG. 19 illustrates an example of a sectional view through a portion of an outer elongate tube, e.g., an outer layer, configured as an outer coil wound tube 1915, having regions of different durometer as described above. In FIG. 19, the outer layer includes a first (e.g., outer) region 1947 over a second (e.g., inner) region 1945. The second (e.g., inner) region 1945 has a durometer that is lower than the durometer of the first (e.g., outer) region 1947 of the outer elongate tube. In this example, the rigidizing layer between the inner and outer elongate tubes is configured to be driven into the lower durometer (e.g., softer) second region of the outer elongate tube. The rigidizing layer (not shown in FIG. 19) may contact with the second inner region of the outer elongate tube and may a somewhat soft. Pressurization (e.g. positive pressure) may provide enhanced rigidization. The outer layer 1915 also includes a reinforcing member 1949 as described above. In some examples the outer layer may also include a torsional stiffening layer (not shown in FIG. 19). The outer region (radially more distant) 1947 and the inner region (radially less distant) 1945 may have different thicknesses or the same thickness. The region between the inner and outer thicknesses may have the same durometer as either the inner region 1945 or the outer region 1947, or a distinct durometer from either of them.

FIGS. 20A-20N schematically illustrate different arrangements of sections through the rigidizable devices described herein. The drawings are not to scale. Each of these typically includes a rigidizing layer, which may be any of the rigidizing layers described herein. These different examples illustrate different configurations that may be used with any of the features or methods described herein, including the application of pressure against the rigidizing layer in different directions, e.g., applying positive pressure to push the rigidizing layer outward, or applying positive pressure to push the rigidizing layer inward. For example, FIG. 20A illustrates a rigidizing device 2200a including an innermost layer 2215a, pressure gap 2212a, a compression layer (e.g., configured as a bladder layer) 2221a that is sealed to the outermost layer 2201a, rigidizing layer 2209a, and outer containment layer 2201a. The rigidizing device 2200a may further include end caps 2292a at the proximal and distal ends thereof to seal the pressure therein. When pressure is supplied to the pressure gap 2212a via inlet 2293a, the bladder layer 222la is pressed against the rigidizing layer 2209a, which in turn is pressed against the outermost layer 2201a, preventing the rigidizing layer from moving (e.g., in some examples preventing sliding of filaments of the rigidizing layer from moving relative to one another).

Referring to FIG. 20H, the rigidizing device 2200j is similar to rigidizing device 2200a except that slip layer 2213j and stiffening layer 2298j are included. Layer 2213j can be a slip layer as described herein, for example comprising a coating film or powder. Layer 2298j can be a stiffening layer that, similar to layers 2201j and 2215j, can include a reinforcement element 2250z. The additional stiffening layer 2298j can work in concert with the inner layer 2215j. For example, the two layers 2215j and 2298j can easily slip past one another (via slip layer 2213j) in the flexible configuration and stick to one another to form a stiff composite structure in the rigid configuration (i.e., when pressure is applied). This layer could be a torsionally stiffening layer. Layer 2298j can be a high durometer elastomeric rubber, for example a TPU or TPE with a durometer greater than or equal to 60 A, 70 A, 80 A or 90 A. When the tube is in a flexible state, layers 2215j and 2298j may easily shear or move with respect to each other (e.g., due to slip layer 2213j) such that the flexibility of the system is lower than it would be if the layers were bonded together. When the tube is in a rigid state (for example, when pressure is applied), layers 2215j, 2298j and 2213j may lock to each other and act like a single bonded layer in order to resist collapse of the wall of the rigidizing device 2200j. Similar to other examples, the rigidizing layer 2205j can push against the outer layer 2201j when pressure is supplied to gap 2212j to rigidize the device 2200j.

Referring to FIG. 20B, rigidizing device 2200b is similar to rigidizing device 2200a except that the pressure gap 2212b is surrounded by a compression layer comprising an everted bladder layer 2221b (or a double-layered bladder), i.e., such that the bladder layer 2221b includes one side that borders the rigidizing layer 2205b and one side that borders the innermost layer 2215b. As pressure is supplied to the pressure gap 2212b (inside of the two sides of the bladder layer 2221b), the bladder layer 2221b can expand both against the innermost layer 2215b and against the rigidizing layer 2209b (which in turn can be pushed against the outermost layer 2201b). In other examples, the apparatus may include dual braids, e.g., one layer outward (2205b) and one layer inward (not shown), that may be pushed against 2215b as pressure is applied. This braid could be one continuous braid (for example, a braid material that is everted), or two separate braids. If it is one continuous braid, it could have a continuous pitch throughout, or different pitches, for example, such that the outer portion and the inner portion are different, or the proximal and distal portions are different.

Referring to FIG. 20C, rigidizing device 2200c is similar to rigidizing device 2200a except that the bladder layer 2221c is sealed to the innermost layer 2215c rather than the outermost layer 2201c. When pressure is supplied to the pressure gap 2212c via inlet 2293c, the compression layer (e.g., bladder layer 2221c) is pressed against the rigidizing layer 2209c, which in turn is pressed against the outermost layer 2201c.

FIG. 20G is similar to FIG. 20C but the compression layer pushes the rigidization layer inward against the ICWT, rather than outward against the OCWT.

Referring to FIG. 20D, rigidizing device 2200d is similar to rigidizing device 2200b except that the innermost layer 2215d is a spring element rather than a coil-wound tube. Because the pressure is in the everted bladder layer 2221d, the inner layer 2215d need not be sealed itself.

Referring to FIG. 20E, rigidizing device 2200e is similar to rigidizing device 2200a except that the innermost layer 2215a is replaced with an inner payload 2294e that is sealed at both the proximal and distal ends and can include a plurality of lumens therein (e.g., a working channel 2291e, a pressure channel 2292e, and a rinse channel 2293e).

Referring to FIG. 20F, rigidizing device 2200f is similar to rigidizing device 2200a except that the rigidizing layer 2209f is inside of the pressure gap 2212f and the compression layer (e.g., bladder layer 2221f) such that pressure supplied to the pressure gap 2212f causes the bladder layer 2221f to push inwards against the rigidizing layer 2209f, which in turn pushes against innermost layer 2215f.

Referring to FIG. 20I, rigidizing device 2200k is similar to rigidizing device 2200a except that an annular ring 2219k, e.g., including fibers and adhesive, is positioned around each of the ends of the rigidizing layer 2209k and bladder layer 2221k to attach the compression (e.g., bladder) layer 2221k to the innermost layer 2215k (and thereby hold pressure within the pressure gap 2212k when pressure is supplied through the inlet 2293k). The annular ring 2219k can, for example, include a high strength fiber, such as Kevlar or Dyneema. Further, the adhesive can be, for example, a cyanoacrylate. In some examples, adhesive can also be placed at the ends between the innermost layer 2215k and the bladder layer 2221k and also encompassing the inlet tube. 201 also includes a secondary inlet 2282. This feature can be active or passive. This feature can be added to any of the other embodiments.

FIG. 20J shows a rigidizing device 2200g with gap inlet 2293g and vent inlet 2223g. Inlet 2293g connects to pressure gap 2212g (via pressure line 2294g). Inlet 2223g connects to gap 2206g around the rigidizing layer 2209g (between bladder 2221g and outermost layer 2201g). The device 2200g can be rigidized in one or more different configurations. In a first rigidizing configuration, pressure can be applied to inlet 2293g while the vent inlet 2223g can be open or vented to atmospheric pressure. The pressure supplied to the pressure gap 2212g through the inlet 2293g can thus push the rigidizing layer 2209g against the outermost layer 2201g, which in turn can force any air in the gap 2206g out through the vent inlet 2223g. Allowing the air to escape through the vent inlet 2223g can enable a tighter mechanical fit between the rigidizing layer 2209g and the outer layer 2201g, thereby strengthening the rigidization of the device 2200g. In a second rigidizing configuration, pressure can be applied to inlet 2293g and a vacuum can be applied to vent inlet 2223g. This may cause the rigidizing device 2200g to become even stiffer than in the first configuration, as the vacuum can assist in moving the rigidizing layer 2209g towards the outer layer 2201g. Further, it removes mass that would therefore not be able to escape into the body, should the device exhibit structural failure during use. The device 2200g can likewise be made flexible in one or more different configurations. In a first flexible configuration, both inlet 2293g and vent inlet 2223g can be opened to atmospheric pressure. This may loosen the rigidizing layer 2209g relative to the outer layer 2201g and cause the rigidizing device 2200g to be flexible as the rigidizing layer 2209g moves freely relative to the outer layer 2201g. In a second flexible configuration, a low pressure (e.g., 5-10% above atmospheric pressure) can be provided to both inlet 2293g and vent inlet 2223g. This may cause the outermost layer 2201g and the innermost layer 2215g to separate slightly, which can provide additionally area for the rigidizing layer 2209g to move freely. As a result, this may cause the rigidizing device 2200g to become even more flexible than in the first rigidizing configuration. Additionally, providing a low pressure above atmospheric pressure in the flexible configuration can allow the rigidizing device 2200g to be introduced into the body with a very small diameter (e.g., such that the pressure gap 2212g is essentially zero) and then the low pressure can be provided to both inlet 2293g and vent inlet 2223g to slightly expand the pressure gap 2212g to provide more room for the rigidizing layer 2209g to move freely.

FIG. 20K shows a rigidizing device 2200h with bellows 2243h connected to pressure line 2294h. Pressure gap 2212h, pressure line 2294h, and bellows 2243h can all be configured to be filled with a sealed pressure transmitting medium, such as distilled water or saline solution or an oil. The pressure transmitting medium may be a radiopaque fluid that advantageously will show the rigidized device more clearly during a procedure using fluoroscopy. The pressure transmitting medium can be added to the rigidizing device immediately before use and/or when the device is being manufactured. In use, activating the actuator 2288h can compress bellows 2243h, thus reducing the volume of pressure medium in the bellows 2243h, which flows through the pressure line 2294h to the pressure gap 2212h, causing a rise in pressure in the pressure gap 2212h and movement of the rigidizing layer 2209h against the outer layer 2201h. The vent inlet 2223h can be open to the atmosphere to allow gas to escape from the space 2206h around the rigidizing layer 2209h. Further, reversing the action of the actuator 2288h can cause the pressure in the pressure gap 2212h to fall as the pressure medium moves back to the bellows 2243h. Actuator 2288h can be, for example, a solenoid, a voice coil, a lead screw, a valve, or a rotary cam. In some examples, the pressure line 2294h can be pinched or flattened to raise the pressure in pressure gap 2212h rather than using bellows 2243h.

FIG. 20L shows a rigidizing device 2200i including sumps 2230i and 2228i respectively. Sumps 2230i and 2228i may comprise a fluid medium, such as water and a gaseous medium such as air. Pressure or vacuum or combinations thereof may be applied to inlets 2293i, 2223i. Using the sump configuration shown may mean that there is no air or gas in the rigidizing device regardless of the pressurization state of each gap 2206i or 2212i (increased pressure, vacuum or atmospheric pressure). In the event that the gaps leaks during a procedure, this may mean that only the fluid medium enters into the patient. This may offer patient protection from gaseous (e.g. air) embolization.

### Indications and Methods for Use

FIG. 21 graphically illustrates several of the regions in which the apparatuses (devices, system) may be used. For example, catheters, sheaths, scopes (e.g., endoscopes), wires, overtubes, cannulas, trocars or laparoscopic instruments may be used in any of these locations and/or a nested pair of devices (one or more of which are rigidizable) may be used. For example, any of the apparatuses described herein may be configured for use in one or more of: the neurovasculature (e.g., aortic arch, subclavian, carotid, vertebral, basilar, posterior cerebral, circle of Willis, middle cerebral, anterior cerebral, etc.), the upper GI tract (mouth esophagus, stomach, pylorus, bile duct and pancreatic duct, etc.), the small bowel (e.g., small intestine, duodenum, jejunum, ilium, etc.), the lower GI tract (rectum, regions of colon, e.g., sigmoid, descending, transverse, ascending, cecum, ileocecal valve, etc.), the urinary tract (urethra, bladder, kidneys, ureters, etc.), the peripheral vasculature (e.g., femoral, iliac, mesenteric, lumbar, renal, celiac trunk, hepatic, thoracic, etc.), the cardiac region (e.g., aorta, right coronary artery, left coronary artery, etc.), the left heart (e.g., aorta, aortic valve, left ventricle, etc.), the right heart (e.g., vena cava, right atrium, left atrium, mitral valve, coronary sinus, tricuspid valve, right ventricle, pulmonary valve, pulmonary vasculature, etc.) and/or the right pulmonary region (e.g., mouth, larynx, trachea, bronchial tree and lobes etc.).

In general, any of the apparatuses (and methods of using them) described herein may be used with, or as part of, a catheter, an endoscope (including, but not limited to colonoscopes, bronchoscope, colposcope, cystoscope, esophagoscope, gastroscope, laparoscope, thoracoscope, enteroscope, etc.), overtube, etc. These apparatuses and methods may be used with a robotic system, including a robotically controlled endoscope. Robotic systems may be steered and/or advanced robotically. In some examples, the robotic system may control the operation (e.g., advancing, retracting, and/or actuating) of one or more tools to be used within an external working channel, including any of the tools or tool pairs described herein. Any of the apparatuses described herein may be used with a robotic system, including a robotic endoscope system.

### Robotic Apparatuses

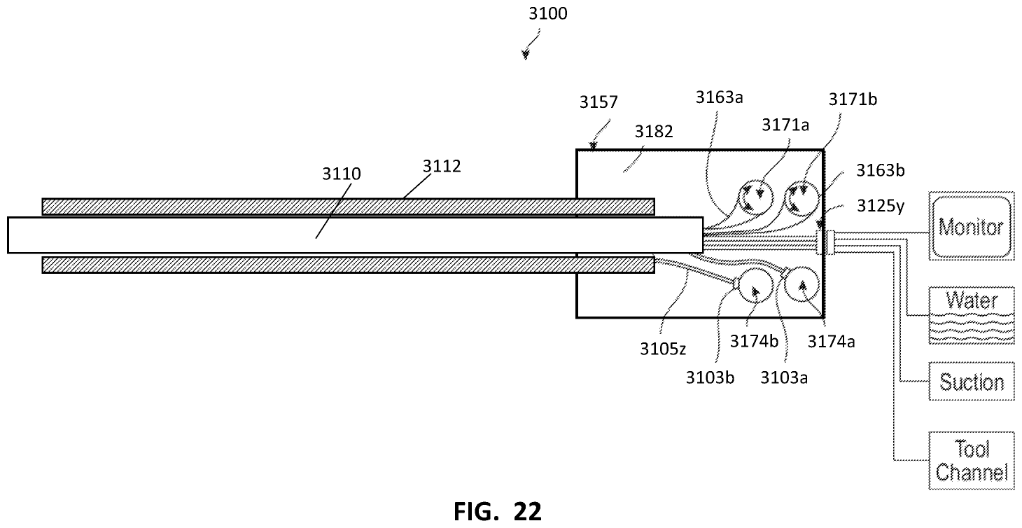

As mentioned above, the rigidizing apparatuses described herein may be configured as part of a robotic system or for use with robotic apparatuses. In some examples the rigidizing apparatus may be configured as an outer tubular member that is robotically controlled, e.g., configured as a robotically controlled overtube and/or endoscope assembly. FIG. 22 shows an exemplary apparatus 3100, including a rigidizing device configured as an overtube 3112; the system may optionally include an inner endoscope 3110. The overtube and inner endoscope can be separately or collectively be robotically controlled or manipulated (e.g., steering, movement, rotation, etc. including in some examples, rigidizing). The overtube and inner endoscope may be configured as illustrated in any of the examples described above, and may have the same general construction, or may be of different constructions. As shown in FIG. 22, the outer overtube 3112 and the inner endoscope 3110 may be terminated together into a common structure, such as a cassette 3157. The outer overtube 3100 can be movable with respect to the endoscope 3110 by rotation of a driver mounted to the cassette 3157. The system may include actuators 3171a, 3171b that may connect to cables 3163a,b respectively, to steer (e.g., bend or deflect) the tip of the endoscope 3110 (and/or outer overtube 3112). Other steering mechanisms (e.g., pneumatics, hydraulics, shape memory alloys, EAP (electro-active polymers), or motors) are also possible. The cassette 3157 can further include bellows 3103a, 3103b that may connect to the pressure gap of the endoscope 3110 and the overtube 3112, respectively to drive fluid through pressure lines 3105z, in variations for either the endoscope and/or the overtube that are configured to rigidize when pressure is applied. As shown in this example, the cassette 3157 can include eccentric cams 3174a,b to control bellows 3103a,b. Alternatively, one or more linear actuators can be configured to actuate the bellows. As another alternative, the devices can be rigidized and de-rigidized through one or more pumps or pressure sources (e.g., via pressure line 3105z).

It should be appreciated that all combinations of the foregoing concepts and additional concepts discussed in greater detail below (provided such concepts are not mutually inconsistent) are contemplated as being part of the inventive subject matter disclosed herein and may be used to achieve the benefits described herein.

The process parameters and sequence of steps described and/or illustrated herein are given by way of example only and can be varied as desired. For example, while the steps illustrated and/or described herein may be shown or discussed in a particular order, these steps do not necessarily need to be performed in the order illustrated or discussed. The various example methods described and/or illustrated herein may also omit one or more of the steps described or illustrated herein or include additional steps in addition to those disclosed.

Any of the methods (including user interfaces) described herein may be implemented as software, hardware or firmware, and may be described as a non-transitory computer-readable storage medium storing a set of instructions capable of being executed by a processor (e.g., computer, tablet, smartphone, etc.), that when executed by the processor causes the processor to control perform any of the steps, including but not limited to: displaying, communicating with the user, analyzing, modifying parameters (including timing, frequency, intensity, etc.), determining, alerting, or the like. For example, any of the methods described herein may be performed, at least in part, by an apparatus including one or more processors having a memory storing a non-transitory computer-readable storage medium storing a set of instructions for the processes(s) of the method.

When a feature or element is herein referred to as being “on” another feature or element, it can be directly on the other feature or element or intervening features and/or elements may also be present. In contrast, when a feature or element is referred to as being “directly on” another feature or element, there are no intervening features or elements present. It will also be understood that, when a feature or element is referred to as being “connected”, “attached” or “coupled” to another feature or element, it can be directly connected, attached or coupled to the other feature or element or intervening features or elements may be present. In contrast, when a feature or element is referred to as being “directly connected”, “directly attached” or “directly coupled” to another feature or element, there are no intervening features or elements present. Although described or shown with respect to one embodiment, the features and elements so described or shown can apply to other embodiments. It will also be appreciated by those of skill in the art that references to a structure or feature that is disposed “adjacent” another feature may have portions that overlap or underlie the adjacent feature.

Terminology used herein is for the purpose of describing particular embodiments only and is not intended to be limiting of the invention. For example, as used herein, the singular forms “a”, “an” and “the” are intended to include the plural forms as well, unless the context clearly indicates otherwise. It will be further understood that the terms “comprises” and/or “comprising,” when used in this specification, specify the presence of stated features, steps, operations, elements, and/or components, but do not preclude the presence or addition of one or more other features, steps, operations, elements, components, and/or groups thereof. As used herein, the term “and/or” includes any and all combinations of one or more of the associated listed items and may be abbreviated as

Spatially relative terms, such as “under”, “below”, “lower”, “over”, “upper” and the like, may be used herein for ease of description to describe one element or feature's relationship to another element(s) or feature(s) as illustrated in the figures. It will be understood that the spatially relative terms are intended to encompass different orientations of the device in use or operation in addition to the orientation depicted in the figures. For example, if a device in the figures is inverted, elements described as “under” or “beneath” other elements or features would then be oriented “over” the other elements or features. Thus, the exemplary term “under” can encompass both an orientation of over and under. The device may be otherwise oriented (rotated 90 degrees or at other orientations) and the spatially relative descriptors used herein interpreted accordingly. Similarly, the terms “upwardly”, “downwardly”, “vertical”, “horizontal” and the like are used herein for the purpose of explanation only unless specifically indicated otherwise.

Although the terms “first” and “second” may be used herein to describe various features/elements (including steps), these features/elements should not be limited by these terms, unless the context indicates otherwise. These terms may be used to distinguish one feature/element from another feature/element. Thus, a first feature/element discussed below could be termed a second feature/element, and similarly, a second feature/element discussed below could be termed a first feature/element without departing from the teachings of the present invention.

In general, any of the apparatuses and methods described herein should be understood to be inclusive, but all or a sub-set of the components and/or steps may alternatively be exclusive and may be expressed as “consisting of” or alternatively “consisting essentially of” the various components, steps, sub-components or sub-steps.

As used herein in the specification and claims, including as used in the examples and unless otherwise expressly specified, all numbers may be read as if prefaced by the word “about” or “approximately,” even if the term does not expressly appear. The phrase “about” or “approximately” may be used when describing magnitude and/or position to indicate that the value and/or position described is within a reasonable expected range of values and/or positions. For example, a numeric value may have a value that is +/−0.1% of the stated value (or range of values), +/−1% of the stated value (or range of values), +/−2% of the stated value (or range of values), +/−5% of the stated value (or range of values), +/−10% of the stated value (or range of values), etc. Any numerical values given herein should also be understood to include about or approximately that value, unless the context indicates otherwise. For example, if the value “10” is disclosed, then “about 10” is also disclosed. Any numerical range recited herein is intended to include all sub-ranges subsumed therein. It is also understood that when a value is disclosed that “less than or equal to” the value, “greater than or equal to the value” and possible ranges between values are also disclosed, as appropriately understood by the skilled artisan. For example, if the value “X” is disclosed the “less than or equal to X” as well as “greater than or equal to X” (e.g., where X is a numerical value) is also disclosed. It is also understood that the throughout the application, data is provided in a number of different formats, and that this data, represents endpoints and starting points, and ranges for any combination of the data points. For example, if a particular data point “10” and a particular data point “15” are disclosed, it is understood that greater than, greater than or equal to, less than, less than or equal to, and equal to 10 and 15 are considered disclosed as well as between 10 and 15. It is also understood that each unit between two particular units are also disclosed. For example, if 10 and 15 are disclosed, then 11, 12, 13, and 14 are also disclosed.

Although various illustrative embodiments are described above, any of a number of changes may be made to various embodiments without departing from the scope of the invention as described by the claims. For example, the order in which various described method steps are performed may often be changed in alternative embodiments, and in other alternative embodiments one or more method steps may be skipped altogether. Optional features of various device and system embodiments may be included in some embodiments and not in others. Therefore, the foregoing description is provided primarily for exemplary purposes and should not be interpreted to limit the scope of the invention as it is set forth in the claims.

The examples and illustrations included herein show, by way of illustration and not of limitation, specific embodiments in which the subject matter may be practiced. As mentioned, other embodiments may be utilized and derived there from, such that structural and logical substitutions and changes may be made without departing from the scope of this disclosure. Such embodiments of the inventive subject matter may be referred to herein individually or collectively by the term “invention” merely for convenience and without intending to voluntarily limit the scope of this application to any single invention or inventive concept, if more than one is, in fact, disclosed. Thus, although specific embodiments have been illustrated and described herein, any arrangement calculated to achieve the same purpose may be substituted for the specific embodiments shown. This disclosure is intended to cover any and all adaptations or variations of various embodiments. Combinations of the above embodiments, and other embodiments not specifically described herein, will be apparent to those of skill in the art upon reviewing the above description.

1.-118. (canceled)

119. A rigidizing device comprising:
a flexible inner tube configured to provide torsional stiffness, wherein the flexible inner tube comprises a first coil wire and a torsional braid;
a flexible outer tube;
a rigidizing layer between the inner and outer tubes;
an inlet configured to attach to a source of positive or negative pressure;
a compression layer configured to be pushed against the rigidizing layer when a positive or negative pressure is applied through the inlet,
wherein the rigidizing device is configured to have a rigid configuration when positive or negative pressure is applied through the inlet and a flexible configuration when positive pressure is released.

120. The device of claim 119, wherein the first coil wire comprises a flat wire helically wound around a length of the flexible inner tube.

121. The device of claim 119, wherein torsional braid comprises a plurality of filaments.

122. The device of claim 119, wherein the torsional braid has a braid angle of greater than 30 degrees.

123. The device of claim 119, wherein the first coil wire and the torsional braid are coupled to each other at discrete regions along the length of the flexible inner tube.

124. The device of claim 119, wherein the first coil wire and the torsional braid are encapsulated within a material.

125. The device of claim 124 wherein the material is a polymeric material.

126. A nested system, comprising:
a first rigidizing device;
a second rigidizing device, the second rigidizing device nested with the first rigidizing device;
wherein the second rigidizing device is configured to have a high torsional stiffness in the unrigidized configuration, so that a distal rotational output can be precisely controlled for a given proximal rotational input.

127. The system of claim 126, further comprising a controller configured to maintain the first rigidizing device in a rigidized configuration while applying torque to the second rigidizing device.

128. The system of claim 126, wherein the second rigidizing device includes a torsional stiffening braid.

129. The system of claim 126, further comprising a lubricious material between the first ridigizing layer device and the second rigidizing device.

130. The system of claim 126, wherein the second rigidizing device is configured so that the output rotation is within +/−15% of the input rotation.

131. The system of claim 126, wherein the second rigidizing device comprises: a flexible inner tube configured to provide torsional stiffness, wherein the flexible inner tube comprises a first coil wire and a torsional braid;
a flexible outer tube;
a rigidizing layer between the inner and outer tubes;
an inlet configured to attach to a source of positive or negative pressure;
a compression layer configured to be pushed against the rigidizing layer when a positive or negative pressure is applied through the inlet,
wherein the rigidizing device is configured to have a rigid configuration when positive or negative pressure is applied through the inlet and a flexible configuration when positive pressure is released.

132. The system of claim 126, further comprising an electronic input device configured to control rotation of the second rigidizing device.

133. The system of claim 132, wherein the electronic input device comprises an actuator configured to rotate the second rigidizing device.

134.-154. (canceled)

155. A rigidizing device comprising:
a flexible inner tube configured to provide torsional stiffness, wherein the flexible inner tube comprises a first coil wire and a torsional braid covered by a flexible polymeric material, wherein the torsional braid has a braid angle of 45 degrees or more;
a flexible outer tube;
a rigidizing layer between the inner and outer tubes, comprising a plurality of lengths of filaments that cross over and under each other;
an inlet configured to attach to a source of pressure;
a compression layer configured to be pushed against the rigidizing layer when a positive or negative pressure is applied through the inlet,
wherein the rigidizing device is configured to have a rigid configuration when a positive or negative pressure is applied through the inlet and a flexible configuration when the positive or negative pressure is released.

156. The device of claim 155, wherein the first coil wire comprises a flat wire helically wound around a length of the flexible inner tube.

157. The device of claim 155, wherein the first coil wire and the torsional braid are coupled to each other.

158. The device of claim 155, wherein the first coil wire and the torsional braid are encapsulated within the polymeric material.

159. The device of claim 155, wherein the first coil wire and the torsional braid are coupled to each other at discrete regions along the length of the flexible inner tube.
